(19) United States
(12) Patent Application Publication
(10) Pub. No.: US 2020/0111574 A1
(43) Pub. Date: Apr. 9, 2020

(54) SYSTEM AND METHOD FOR DERIVING PERSONALIZED CARDIOVASCULAR DISEASE RISK ASSESSMENTS

(71) Applicant: The University Of Trinidad And Tobago, Arima (TT)

(72) Inventors: Amalia Hosein, Cunupia (TT); Natasha Ramroop Singh, Tacarigua (TT); Valerie Stoute, Trinidad (TT); Samantha Chadee, Trinidad (TT); Steve E. Humphries, London (GB)

(21) Appl. No.: 16/150,972

(22) Filed: Oct. 3, 2018

(51) Int. Cl. G16H 50/30; G16H 10/60

(52) U.S. Cl. CPC G16H 50/30; G06F 19/28; G16H 10/60

(57) ABSTRACT

A method for deriving a personalized cardiovascular disease (CVD) risk assessment for an individual, via a computing system, wherein the computing system comprises: a processor operable to control the computing system, data storage operatively coupled to the processor, wherein the data storage is configured to store a plurality of background data, a plurality of phenotypic measurement data, and combinations thereof.

### BACKGROUND

This present disclosure is directed to bioinformatics and statistical inference, focusing on cardiovascular disease risk prediction. The system and method assess risks for cardiovascular disease, based on genetic, environmental, and behavioral risk factors. Certain embodiments may, for example, provide early medical diagnostic devices. For instance, certain embodiments provide diagnostic computerized medical devices that can detect early tendencies of developing cardiovascular disease. Moreover, certain embodiments may provide methods and systems to capture genetic, environmental, and behavioral characteristics associated with cardiovascular disease.

Cardiovascular diseases (CVD) are currently the leading cause of mortality worldwide. It is estimated that 31% of all annual deaths worldwide (16.7 million people) are due to CVD, with 80% of all CVD deaths due to heart attacks and strokes. For example, in Trinidad and Tobago, the southernmost Caribbean islands, CVDs account for 32% of all deaths, one of the highest in the Americas region. The major risk factors associated with CVD are cigarette smoking, unhealthy diet, physical inactivity, hypertension, diabetes and high blood cholesterol. Modification of these risk factors in combination with improved medical therapies have successfully reduced mortality and morbidity in people with diagnosed and undiagnosed cardiovascular disease. The decision about whether to initiate specific preventive action and with what degree of intensity is guided by estimation of the risk for such a vascular event. The risk is determined from prediction charts that accompany specific guidelines that allow treatment to be targeted according to simple predictions of absolute CVD risk. The main CVD risk prediction algorithms are guided by the Framingham heart study in the United States (Kannel, McGee, & Gordon, 1976; Sullivan, Massaro, & D'Agostino, 2004), SCORE study done in Europe (Conroy et al., 2003) and the QRISK2 score used in the United Kingdom (Hippisley-Cox et al., 2007), specific for their respective populations.

In Trinidad and Tobago, five risk factors were identified that can lead to Chronic Non-Communicable Diseases. These five factors were determined using the Pan American STEPS Survey which is smoking, obesity, elevated blood pressure, poor diet and a lack of exercise. A person with a “raised CVD risk profile” is determined by the presence of any three of the five listed risk factors. In 2015, the WHO and the International Society for Hypertension (ISH) identified 14 different country-specific risk prediction charts for specific regions to establish the 10-year risk of having a CVD event.

Even with these risk models, the incidence, as well as the prevalence of CVD, is very high and estimates suggest that CVD will remain the major cause of death by 2030. Clinically, a substantial fraction of high-risk subjects does not reach treatment goals for major risk factors, highlighting the problem of markedly varied treatment responses among individuals and consequently, respective populations. In addition, finding the “concealed” high-risk subjects that are overlooked by current risk assessments constitutes another challenge if CVD incidence is to be significantly reduced.

As is the case for most diseases, family history is a well-established “fixed” risk factor for CVD suggesting a substantial genetic component. This genetic contribution is evidenced by familial aggregation of the disease, and family and twin studies have proposed that genetic factors explain 17 to 61% of the observed variation in CVD mortality or morbidity in the study populations. Genome-wide association studies (GWASs) have uncovered several common genetic variants (single-nucleotide polymorphisms [SNPs]) that are robustly associated with Myocardial Infarction (MI), Coronary Heart Disease (CHD) and CVD risk factors.

Ethnicity has been well established worldwide and in the Caribbean as a non-modifiable CVD risk factor, however, its impact has yet to be determined. It is important to include the unique ethnicity and consequent genetic variations into the prevention and treatment strategies for CVD to reduce morbidity and mortality within certain populations, cultures, regions, and the like.

### SUMMARY

The following presents a simplified overview of the example embodiments in order to provide a basic understanding of some embodiments of the example embodiments. This overview is not an extensive overview of the example embodiments. It is intended to neither identify key or critical elements of the example embodiments nor delineate the scope of the appended claims. Its sole purpose is to present some concepts of the example embodiments in a simplified form as a prelude to the more detailed description that is presented herein below. It is to be understood that both the following general description and the following detailed description are exemplary and explanatory only and are not restrictive.

The system and method of the present disclosure are directed to deriving a personalized cardiovascular disease (CVD) risk assessment for an individual based on the individual's background, phenotypic measurement data, and genetic information. An individual's CVD risk is determined based in part on the individual's ethnicity, cultural background, sociodemographic profile, and the like. The CVD risk assessment may provide short term/long term disease risks and prognosis and support for complex health decisions.

In accordance with the disclosure presented herein, there is provided a method for deriving a personalized cardiovascular disease (CVD) risk assessment for an individual, via a computing system. The computing system comprises a processor operable to control the computing system, data storage operatively coupled to the processor, wherein the data storage is configured to store a plurality of background data, a plurality of phenotypic measurement data, and combinations thereof, and an input/output device operatively coupled to the processor, wherein the input/output device is configured to receive a plurality of data for transmission to the processor, wherein the input/output device is configured to transmit a plurality of data generated by the processor. The computing system further comprises a profile classification component operatively coupled to the processor and controlled in part by the processor, wherein the profile classification component is configured to determine a profile classification for the individual, a risk factor profile component operatively coupled to the processor and controlled in part by the processor, wherein the risk factor profile component is configured to generate a risk factor profile for the individual, and a risk prediction component operatively coupled to the processor and controlled in part by the processor, wherein the risk prediction component is configured to generate a personalized CVD risk assessment for the individual.

The method comprises receiving, via the input/output device, a plurality of selected background data associated with the individual and transmitting at least a portion of the plurality of background data to the profile classification component, and receiving, via the input/output device, a plurality of phenotypic measurement data associated with the individual and transmitting at least a portion of the phenotypic measurement data to the profile classification component. The method also comprises determining, by the profile classification component, based on at least a portion of the background information and the phenotypic measurement data, a profile classification for the individual and generating profile classification data therefrom, and transmitting, via the input/output device, the profile classification data to the risk factor profile component. The method then determines, by the risk factor profile component, based on the profile classification data, at least one individual risk factor and associated risk factor weight to be included in the personalized CVD risk assessment, and generates a personalized risk factor profile for the individual. The method further comprises selectively integrating, by the risk prediction component, at least a portion of the background data and phenotypic measurement data in accordance with the personalized risk factor profile to generate integrated risk data, and subjecting, by the risk prediction component, the integrated risk data to risk prediction function to generate a personalized CVD risk assessment for the individual.

In one embodiment, the selected background data is selected from the group consisting of sociodemographic data, personal and family medical history data, medication usage, diet and physical activity data, behavioral data, and combinations thereof. In another embodiment, the phenotypic measurement data is selected from the group consisting of biomedical record data, health care record data, bioassay data, medical imaging data, blood analysis data, metabolic test data, physiologic data, and combinations thereof.

In one embodiment, the method further comprises receiving, via the input/output device, a plurality of genetic data associated with the individual and transmitting at least a portion of the genetic data to the profile classification component. In such embodiment, the data storage is operable to store a plurality of genetic data, and the profile classification for the individual is determined based on at least a portion of the background information, the phenotypic measurement data, and the genetic data. In a preferred embodiment, the genetic data is selected from the group consisting of genotype data, structural variant data, sequence data, and combinations thereof. In another preferred embodiment, at least a portion of the background data, phenotypic data, and genetic data are selectively integrated by the risk prediction component in accordance with the personalized risk factor profile to generate integrated risk data.

In another embodiment, the computing system further comprises a cardiac health recommendation component operatively connected to the processor and controlled in part by the processor, wherein the cardiac health recommendation component is configured to generate a plurality of cardiac health recommendations. In such embodiment, the method comprises transmitting, via the input/output device, the personalized CVD risk assessment to the cardiac health recommendation component, and determining, by the cardiac health recommendation component, based on the personalized CVD risk assessment, at least one cardiac health regime.

In one embodiment, the integrated risk data is subjected to a logistic regression function to generate a personalized CVD risk assessment for the individual. In another embodiment, the integrated risk data is subjected to a discriminant analysis function to generate a personalized CVD risk assessment for the individual.

In accordance with the disclosure provided herein, there is provided a system for deriving a personalized cardiovascular disease (CVD) risk assessment for an individual. The system comprises a processor operable to control the system, data storage operatively coupled to the processor, wherein the data storage is configured to store a plurality of background data, a plurality of phenotypic measurement data, and combinations thereof, and an input/output device operatively coupled to the processor, wherein the input/output device is configured to receive a plurality of data for transmission to the processor, wherein the input/output device is configured to transmit a plurality of data generated by the processor. The system further includes a profile classification component operatively coupled to the processor and controlled in part by the processor, wherein the profile classification component is configured to determine a profile classification for the individual. The system also includes a risk factor profile component operatively coupled to the processor and controlled in part by the processor, wherein the risk factor profile component is configured to generate a risk factor profile for the individual. The system further comprises a risk prediction component operatively coupled to the processor and controlled in part by the processor, wherein the risk prediction component is configured to generate a personalized CVD risk assessment for the individual.

The input/output device is operable to receive a plurality of selected background data associated with the individual and transmit at least a portion of the plurality of background data to the profile classification component. The input/output device is also operable to receive a plurality of phenotypic measurement data associated with the individual and transmit at least a portion of the phenotypic measurement data to the profile classification component.

The profile classification component is operable to receive at least a portion of the selected background data and the phenotypic measurement data from the input/output device. The profile classification component is also operable to determine, based on at least a portion of the background information and the phenotypic measurement data, a profile classification for the individual and generating profile classification data therefrom, and transmit, via the input/output device, the profile classification data to the risk factor profile component.

The risk factor profile component is operable to receive the profile classification data from the input/output device and determine, based on the profile classification data, at least one individual risk factor and associated risk factor weight to be included in the personalized CVD risk assessment, and generate a personalized risk factor profile for the individual. The risk factor profile component is also operable to transmit, via the input/output device, the personalized risk factor profile to the risk prediction component.

The risk prediction component is operable to receive the personalized risk factor profile from the input/output device, and selectively integrate at least a portion of the background data and phenotypic measurement data in accordance with the personalized risk factor profile to generate integrated risk data. The risk prediction component is also operable to subject the integrated risk data to risk prediction function to generate a personalized CVD risk assessment for the individual.

In one embodiment, the selected background data is selected from the group consisting of sociodemographic data, personal and family medical history data, medication usage, diet and physical activity data, behavioral data, and combinations thereof. In another embodiment, the phenotypic measurement data is selected from the group consisting of biomedical record data, health care record data, bioassay data, medical imaging data, blood analysis data, metabolic test data, physiologic data, and combinations thereof.

In one embodiment, the input/output device is further operable to receive a plurality of genetic data associated with the individual and transmit at least a portion of the genetic data to the profile classification component. In such embodiment, the data storage is operable to store a plurality of genetic data, and the profile classification for the individual is determined based on at least a portion of the background information, the phenotypic measurement data, and the genetic data. In a preferred embodiment, the genetic data is selected from the group consisting of genotype data, structural variant data, sequence data, and combinations thereof. In another preferred embodiment, the risk prediction component is operable to selectively integrate at least a portion of the background data, phenotypic data, and genetic data in accordance with the personalized risk factor profile to generate integrated risk data.

In another embodiment, the system further comprises a cardiac health recommendation component operatively connected to the processor and controlled in part by the processor, wherein the cardiac health recommendation component is configured to generate a plurality of cardiac health recommendations. In such embodiment, the input/output device is operable to transmit the personalized CVD risk assessment to the cardiac health recommendation component, and the cardiac health recommendation component is operable to determine, based on the personalized CVD risk assessment, at least one cardiac health regime.

In one embodiment, the risk prediction component is operable to subject the integrated risk data to a logistic regression function to generate a personalized CVD risk assessment for the individual. In another embodiment, the risk prediction component is operable to subject the integrated risk data to a discriminant analysis function to generate a personalized CVD risk assessment for the individual.

Still other advantages, embodiments, and features of the subject disclosure will become readily apparent to those of ordinary skill in the art from the following description wherein there is shown and described a preferred embodiment of the present disclosure, simply by way of illustration of one of the best modes best suited to carry out the subject disclosure As it will be realized, the present disclosure is capable of other different embodiments and its several details are capable of modifications in various obvious embodiments all without departing from, or limiting, the scope herein. Accordingly, the drawings and descriptions will be regarded as illustrative in nature and not as restrictive.

### BRIEF DESCRIPTION OF THE DRAWINGS

The drawings are of illustrative embodiments. They do not illustrate all embodiments. Other embodiments may be used in addition or instead. Details which may be apparent or unnecessary may be omitted to save space or for more effective illustration. Some embodiments may be practiced with additional components or steps and/or without all of the components or steps which are illustrated. When the same numeral appears in different drawings, it refers to the same or like components or steps.

FIG. 1 is an overview of exemplary systems and methods for deriving personalized cardiovascular disease risk assessments according to the present disclosure.

FIG. 2 is a block diagram illustrating an example system environment for deriving personalized cardiovascular disease risk assessments according to the present disclosure.

FIG. 3 is a chart illustrating many of the risk factors associated with cardiovascular disease.

FIG. 4 illustrates the Relative Operating Characteristic (ROC) plot for three established CVD risk prediction models with respect to a sample population.

FIG. 5 illustrates a CHAID tree diagram for a Training and Test split sample for a model using the presence of selected predictors in accordance with the present disclosure.

FIG. 6 illustrates example risk scores from a logistic regression model according to the present disclosure of 10 significant predictor variables from a sample population for non-cardiovascular disease participants and cardiovascular disease participants.

FIG. 7 illustrates example risk scores from a discriminant analysis model according to the present disclosure using 12 significant predictor variables from a sample population for non-cardiovascular disease participants and cardiovascular disease participants.

FIG. 8 illustrates one embodiment of a cardiac health recommendation protocol according to the present disclosure.

FIG. 9 illustrates ROC curves with AUROC values from 5 risk models evaluated to discriminate between non-cardiovascular disease participants and cardiovascular disease participants in a sample population.

FIG. 10 illustrates a percentage distribution of persons categorized by three established risk models into different risk levels for non-cardiovascular disease and cardiovascular disease groups.

### DETAILED DESCRIPTION OF THE ILLUSTRATIVE EMBODIMENTS

Before the present methods and systems are disclosed and described, it is to be understood that the methods and systems are not limited to specific methods, specific components, or to particular implementations. It is also to be understood that the terminology used herein is for the purpose of describing particular embodiments only and is not intended to be limiting.

As used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are in relation to the other endpoint, and independently of the other endpoint.

“Optional” or “optionally” means that the subsequently described event or circumstance may or may not occur, and that the description includes instances where said event or circumstance occurs and instances where it does not.

Throughout the description and claims of this specification, the word “comprise” and variations of the word, such as “comprising” and “comprises,” means “including but not limited to,” and is not intended to exclude, for example, other components, integers or steps. “Exemplary” means “an example of” and is not intended to convey an indication of a preferred or ideal embodiment. “Such as” is not used in a restrictive sense, but for explanatory purposes.

Disclosed are components that may be used to perform the disclosed methods and systems. These and other components are disclosed herein, and it is understood that when combinations, subsets, interactions, groups, etc. of these components are disclosed that while specific reference of each various individual and collective combinations and permutation of these may not be explicitly disclosed, each is specifically contemplated and described herein, for all methods and systems. This applies to all embodiments of this application including, but not limited to, steps in disclosed methods. Thus, if there are a variety of additional steps that may be performed it is understood that each of these additional steps may be performed with any specific embodiment or combination of embodiments of the disclosed methods.

The present methods and systems may be understood more readily by reference to the following detailed description of preferred embodiments and the examples included therein and to the Figures and their previous and following description.

As will be appreciated by one skilled in the art, the methods and systems may take the form of an entirely hardware embodiment, an entirely software embodiment, or an embodiment combining software and hardware embodiments. Furthermore, the methods and systems may take the form of a computer program product on a computer-readable storage medium having computer-readable program instructions (e.g., computer software) embodied in the storage medium. More particularly, the present methods and systems may take the form of web-implemented computer software. Any suitable computer-readable storage medium may be utilized including hard disks, CD-ROMs, optical storage devices, or magnetic storage devices.

Embodiments of the methods and systems are described below with reference to block diagrams and flowchart illustrations of methods, systems, apparatuses and computer program products. It will be understood that each block of the block diagrams and flowchart illustrations, and combinations of blocks in the block diagrams and flowchart illustrations, respectively, may be implemented by computer program instructions. These computer program instructions may be loaded onto a general-purpose computer, special purpose computer, or other programmable data processing apparatus to produce a machine, such that the instructions which execute on the computer or other programmable data processing apparatus create a means for implementing the functions specified in the flowchart block or blocks.

These computer program instructions may also be stored in a computer-readable memory that may direct a computer or other programmable data processing apparatus to function in a particular manner, such that the instructions stored in the computer-readable memory produce an article of manufacture including computer-readable instructions for implementing the function specified in the flowchart block or blocks. The computer program instructions may also be loaded onto a computer or other programmable data processing apparatus to cause a series of operational steps to be performed on the computer or other programmable apparatus to produce a computer-implemented process such that the instructions that execute on the computer or other programmable apparatus provide steps for implementing the functions specified in the flowchart block or blocks.

Accordingly, blocks of the block diagrams and flowchart illustrations support combinations of means for performing the specified functions, combinations of steps for performing the specified functions and program instruction means for performing the specified functions. It will also be understood that each block of the block diagrams and flowchart illustrations, and combinations of blocks in the block diagrams and flowchart illustrations, may be implemented by special purpose hardware-based computer systems that perform the specified functions or steps, or combinations of special purpose hardware and computer instructions.

In the following description, certain terminology is used to describe certain features of one or more embodiments. For purposes of the specification, unless otherwise specified, the term “substantially” refers to the complete or nearly complete extent or degree of an action, characteristic, property, state, structure, item, or result. For example, in one embodiment, an object that is “substantially” located within a housing would mean that the object is either completely within a housing or nearly completely within a housing. The exact allowable degree of deviation from absolute completeness may in some cases depend on the specific context. However, generally speaking, the nearness of completion will be so as to have the same overall result as if absolute and total completion were obtained. The use of “substantially” is also equally applicable when used in a negative connotation to refer to the complete or near complete lack of an action, characteristic, property, state, structure, item, or result.

As used herein, the terms “approximately” and “about” generally refer to a deviance of within 5% of the indicated number or range of numbers. In one embodiment, the term “approximately” and “about”, may refer to a deviance of between 0.001-10% from the indicated number or range of numbers.

Various embodiments are now described with reference to the drawings. In the following description, for purposes of explanation, numerous specific details are set forth in order to provide a thorough understanding of one or more embodiments. It may be evident, however, that the various embodiments may be practiced without these specific details. In other instances, well-known structures and devices are shown in block diagram form to facilitate describing these embodiments.

In various implementations, there may be provided a system and methods for deriving a personalized cardiovascular disease (CVD) risk assessment for an individual based on the individual's background, phenotypic measurement data, and genetic information. An individual's CVD risk is determined based in part on the individual's ethnicity, cultural background, sociodemographic profile, and the like. The CVD risk assessment may provide short term/long term disease risks and prognosis and support for complex health decisions.

Certain embodiments may relate generally to assessing risks for cardiovascular disease, based on genetic, environmental, and behavioral risk factors. Certain embodiments may, for example, provide early medical diagnostic devices. For example, certain embodiments provide diagnostic computerized medical devices that can detect early tendencies of developing cardiovascular disease. Moreover, certain embodiments may provide methods and systems to capture genetic, environmental, and behavioral characteristics associated with cardiovascular disease.

Statistical analysis may be used to understand the correlation and, in certain instances, causality of identified factors in developing cardiovascular disease. Statistical analysis may also be used to develop risk assessment models composed of independent risk factors. Subgroup analysis by ethnicity, cultural background, or any other sociodemographic classification may be used to model genetic, environmental, and behavioral risks separately for different sociodemographic profiles.

Multiple computer-implemented processes may be implemented. These processes may be integrated together or modularized, such that various combinations of the modules may be implemented in different embodiments.

### I. Cardiovascular Disease (CVD) Risk Assessment Systems and Methods

The World Health Organization reports that 16.7 million annual deaths worldwide are due to cardiovascular diseases (CVD). Death from CVD is predicted to rise to 23 million per annum by 2030. Globally, among the CVD, coronary heart disease is responsible for 43% deaths followed by strokes (36%), hypertensive heart disease (5%), inflammatory and rheumatic heart disease are 2% each while the remaining 12% are for non-specific CVD related conditions. Although mortality from CVD has declined considerably from the 1970s, it remains the number one cause of mortality from chronic diseases.

The major CVD include coronary heart disease, cerebrovascular disease, heart failure, rheumatic heart disease and congenital heart disease. It is the leading cause of death worldwide with predictions sending it from 16.7 million (in 2011) to 23 million per annum by 2030. Globally, coronary heart disease is responsible for 43% deaths followed by stroke (36%), hypertensive heart disease (5%), inflammatory heart disease (2%) rheumatic heart disease 2% and other CVD causes accounting for 11% of death. Although mortality from CVD has been decreasing, it is still the number one cause of mortality among chronic diseases.

Coronary Heart Disease (CHD) and stroke, are the two most common forms of CVD. Both conditions are mainly caused by atherosclerosis, a condition where arteries become narrowed by a gradual build-up of fatty material (i.e., atheroma) within artery walls. When the arteries become too narrow and there is inadequate oxygen-rich blood delivered to the heart, it causes angina, manifested by a pain or discomfort in the chest. When an atheroma or part of it in the arteries breaks away, it causes clotting in the circulation and cutting off the supply of oxygen-rich blood to the heart muscle, leading to myocardial infarction (MI), commonly known as heart attack. When the blood clot blocks an artery that carries blood to the brain, it causes an ischemic stroke. Another form of stroke is a haemorrhagic stroke, caused by the rupture of a blood vessel in the brain. Worldwide, the mortality data from Coronary Heart Disease (CHD) is well documented, with Turkmenistan having the largest mortality rate (400 deaths annually per 100,000 persons) compared to Kiribati with a rank of 192 and the lowest death rate annually (22 deaths annually per 100,000).

Risk factors are important for assessing disease risk and consequently managing disease prevention. Risk factors are usually first identified through epidemiological studies, such as the Framingham Heart Study (FHS). The criteria for being an established CVD risk factors include a significant independent impact on the risk of CVD, a high prevalence in many populations, and a reduced level of CVD by the treatment and control of the risk factor.

Low-Density Lipoprotein (LDL) was one of the first established risk factors for CVD. The decrease in mortality from CVD since 1980s was closely associated with lowering underlying risk factors especially LDL, which accounted for more than one-third of the observed decrease in mortality from Coronary Heart Disease. In addition, CVD risk factors were identified as dyslipidemia, hypertension, obesity, smoking, and physical inactivity. A review of the literature examining the association between alcohol and CVD outcome showed that light to moderate alcohol consumption was associated with a reduced risk of multiple CVD outcomes.

Other risk factors included obesity measured by increased Body Mass Index, presence of diabetes, metabolic syndrome and increased waist-hip ratios. CVD risk factors also included inflammatory markers especially C-reactive protein, haemostasis markers such as fibrinogen, white blood cell count (WBC), homocysteine, and uric acid.

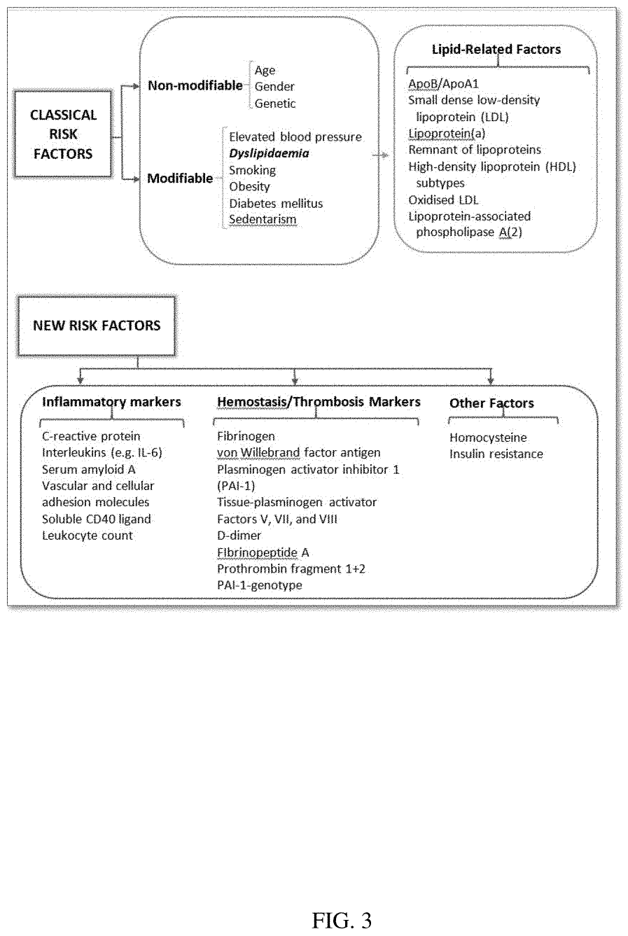

All of the risk factors above are involved in initiating atherosclerosis. FIG. 3 illustrates both the classical risk factors and the new risk factors associated with cardiovascular disease. Most CVDs can be prevented by addressing modifiable risk factors such as smoking, unhealthy diet and physical inactivity, hypertension, and dyslipidaemia. Risk factors have been used to estimate the onset of both non-fatal and fatal cardiovascular events through the calculation of a risk score.

Age and Sex are two of the major non-modifiable predictors of CVD outcome. Persons with increasing age show a greater tendency for a CVD outcome. Nonetheless, it can be difficult to separate the aging process from a simultaneous age-related disease. Traditional clinical measures of cardiovascular function may underestimate the effects of age on the cardiovascular system, explaining in part why age remains such a dominant factor. Age is associated with increased co-morbidity and can influence behavioural risk factors.

In the US, the prevalence of CVD and other chronic conditions increased with age, with the highest rates occurring among the 85 years and older. In men, prevalence rates increased between the two younger groups, but the oldest group had lower than expected rates for coronary heart disease, cerebrovascular disease, hypertension, and chronic lung disease.

In the Caribbean, there are strong trends which showed that older persons are more likely to have high blood pressure, atherosclerosis and poor pulmonary function for both sexes across the age range. Studies have shown that the admission of strokes in Trinidad and Tobago hospitals over a 12 month period, indicated an expected age effect (Mahabir, Bickram, & Gulliford, 1998). In the younger age groups, admission rates were higher in women than in men, but at older ages, admission rates were similar for both sexes. In those under 65 years of age, admission rates were higher in Indo-Trinidadians than in Afro-Trinidadians, while at older ages admission rates were similar in these two ethnic groups.

Sex is also an important factor in CVD outcome. In the United States and the United Kingdom populations, the prevalence of coronary heart disease mortality was higher among men compared to women. Although CVD is the biggest cause of mortality among women, the incidence rates are comparable with those of men 10 years younger. A single study conducted in a mixed ethnic Caribbean population found that 5 year mortality rate from strokes was lower in women compared to men (Incidence 134 vs. 185/100,00 per year) and was in higher in the 35-74 years old group compared to those 75-84 years for both men and women. Studies on Caribbean populations showed that the incidence of coronary heart disease increases with age but only in men.

Ethnicity (unlike race) is a construct that encompasses both genetic and cultural differences. Individuals with different ethnic backgrounds tend to live in distinct regions and societies, and variations in disease rates by ethnicity are also intertwined with geographic differences. Further, while a specific ethnic group in one location can adopt a certain lifestyle, that same ethnic group in another location may adopt a substantially different lifestyle.

Ethnicity has been well established worldwide as a CVD risk factor Migrants of South Asian descent (viz. East Indians) worldwide have elevated risks of morbid and mortal events because of ischaemic heart disease (IHD). In the UK mortality from IHD in both South Asian men and women is 1.5 times that of the general population.

Interestingly, African-Caribbeans (persons with African heritage from the Caribbean) have been identified as a separate ethnic group in analyses performed in the UK and US Data on Indo-Caribbeans (viz. East Indians from the Caribbean) with other South Asian groups is limited and as a group, there has not been any significant differentiation among South Asians. Similarly, for the Mixed-Caribbeans, which are persons with both African and South Asian heritage from the Caribbean, there is limited data for these group of persons related specifically to CVD.

In the UK, although the Afro-Caribbeans have 1.5-2.5 times greater risk of stroke, they are at a significantly lower risk of heart disease compared to the majority of the population. The majority of the strokes are thrombotic, as observed in Europeans. Notably, post-stroke survival is better among Africans presumably because of the protection from IHD. Francis et al. (2015) in a recent systematic review of the literature looked at disparities in CVD among the Afro-Caribbeans, where he concluded that Coronary Heart Disease (CHD) and Peripheral Arterial Disease (PAD) were less prevalent among Afro-Caribbeans compared to Caucasian and South East Asian ethnic groups. He reported that CHD ranged from 0-7% in Afro-Caribbean to 2-22% in Caucasians and strokes were more common among Afro-Caribbeans than other ethnicities.

Risk prediction is defined as actions directed to avoid illness and promoting health so as to reduce the need for secondary and tertiary healthcare. The modifiable (e.g. cholesterol, weight, blood pressure) and non-modifiable (e.g. age, sex, family history) risk factors have been used to create risk prediction algorithms in order to determine a 10-year risk of having a CVD event. The main risk models are the Framingham risk score (Wilson et al., 1998), the ASSIGN score (H Tunstall-Pedoe, 2006), the Systematic Coronary Risk Evaluation (SCORE) risk charts (Graham et al., 2007) and the QRISK2 score (Hippisley-Cox et al., 2007). There are differences among these approaches (see Table). For instance, the Framingham risk score is based on data from a single community, while the QRISK2 model was limited to persons from the United Kingdom and the SCORE risk charts are based on data from 12 European countries.

TABLE 1
Differences between 4 CVD risk prediction models

| | FRAM | SCORE | ASSIGN | QRISK2 |
|---|---|---|---|---|
| Age | Yes | Yes | Yes | Yes |
| Sex | Yes | Yes | Yes | Yes |
| Cholesterol levels | Yes | No | Yes | Yes |
| Smoking | Yes | No | Yes | Yes |
| BP treatment | Yes | No | No | Yes |
| Diabetes | Yes | No | Yes | Yes |
| Family History | No | No | Yes | Yes |
| Social deprivation | No | No | Yes | Yes |
| Ethnicity | No | No | No | Yes |
| Reproducibility | Yes | Yes | Yes | Yes |
| Generalisability | — | — | — | Yes |
| Statistical validity | Yes | Yes | Yes | Yes |
| Face validity | Yes | No | No | Yes |

Source: D'Agostino et al. (2008); Graham et al. (2007); Hippisley-Cox et al. (2007); Hugh Tunstall-edoe (2011)

These epidemiologic risk models, however, do not include differences that occur among regions and countries due to different lifestyles, life expectancy, and genetic predisposition. Thus, it is expected that these risk prediction algorithms should evolve over time. Currently, persons with <10%, 10-20%, and >20% CVD risks scores are considered low, intermediate and high risk, respectively. CVD 10-year risk scores are important in determining the prescription of preventive drugs such as those lowering blood pressure or cholesterol levels. A 10-year CVD risk of 20% or more, allows for treatment with statin HTA which has resulted in a large number of non-fatal events. This highlights the necessity, for accurate and effective CVD risk prediction systems and methods.

Most CVD risk factors have an underlying genetic component, and as such, there is a need to identify genetic markers and their function. Heritable factors contribute to 30%-60% risk of coronary artery disease (CAD) with Atherosclerosis Coronary Artery Disease (CAD) having a strong heritable component (estimates as high as >50%). Families with CAD (14% of US population) account for 72% of early CAD cases.

Family history is a well-established risk factor for CVD, and this heritability holds true independent of other known CVD risk factors, which highlights a substantial underlying genetic component. A number of rare Mendelian forms of CVD (viz. the LDL-receptor mutations and other mutations that cause familial monogenic hypercholesterolemia) have previously been identified using linkage analysis and subsequent genetic refinement. Later, direct exome sequencing was utilized for finding genetic variants for other Mendelian-type CVD, such as rare forms of genetic hypertension.

A single nucleotide polymorphism (SNP) is one form of genetic variation that can be used for genetic risk calculation. SNP association data may be extracted from large scale genome-wide association studies (GWAS) for the disease and/or condition of interest. For common multifactorial forms of CVD in the general population, a breakthrough occurred in 2007 when the first GWASs for CAD were published. These studies showed that the SNPs at chromosome 9p21 with risk allele frequencies of approximately 50% in Caucasian populations were strongly associated with Myocardial infarction (MI) and Cardiovascular Artery Disease (CAD), with an odds ratio (OR) of approximately 1.30 per risk allele.

In 2013, GWAS identified 15 new SNPs that associate with CAD suggesting that 50 common SNPs associating with CAD and/or MI have been further reported. Whilst some of these SNPs are associated with traditional CVD risk factors, such as lipids and hypertension, the majority of the reported CAD/MI SNPs did not show any such associations, with many SNPs located in DNA regions not previously thought be involved in CVD. Altogether, the common genetic variants discovered explained approximately 10% of the heritability for CAD. In addition, there are numerous SNPs associated with risk factors for CAD (of which some but not all also show direct associations with the hard endpoint of a disease).

Prior to the Human Genome Project, many genes associated with mendelian CVD had been identified. These forms of CVD were rare and constitute a minority of clinical CVD. Genetically, they were simple in that a mutation in a single gene is sufficient to cause disease, so mendelian disease was referred to as monogenic such as forms of premature myocardial infarction, dilated and hypertrophic cardiomyopathy, heart failure, arrhythmogenic right ventricular dysplasia, the long-QT syndrome, and aortic aneurysms. The large majority of CVD, however, are polygenic, with both heritable and environmental contributions.

This genetic abundance is a result of genome-wide association studies that have identified a large number of new loci associated with CVD risk factors, subclinical indexes, and disease endpoints which have provided further insights into the biologic pathways that underlie the disease. Many of these loci show association with CVD across groups of differing ancestry, and most CVD traits are influenced by a large number of loci. However, the limitations of genome-wide association studies have prevented immediate translation of these findings into clinical practice, since each variant has a very small effect and has not been proven useful for prediction. Moreover, the implicated variants are rarely themselves the causal variants; rather, they are linked to the true causal variants, and identification of the latter usually warrants a great deal of additional work. Table 2 gives the SNPedia characterisation of 19 SNPs analysed with respect to cardiovascular disease according to the present disclosure, and illustrates the effect of each variant as it relates to disease magnitude.

TABLE 2
19 SNPs from GWAS study

| | | | | Risk | Major Ethnic |
|---|---|---|---|---|---|
| Study | Chromosome | SNP | Location | Allele | Group |
| (Bennet, Di Angelantonio, | APOE112 | rs429358 | C112R | C | European, East |
| Ye, & et al., 2007) | | | | | Indian |
| (Bennet et al., 2007) | APOE158 | rs7412 | C158R | C | European, East |
| | | | | | Indian |
| (Benn, Nordestgaard, | PCSK9 | rs11591147 | R46L | G | European |
| Grande, Schnohr, & | | | | | |
| Tybjaerg-Hansen, 2010) | | | | | |
| (Casas, Cooper, Miller, | eNOS | rs1799983 | E289D | G | European |
| Hingorani, & Humphries, | | | | | |
| 2006) | | | | | |
| (Sarwar N, 2010) | APOA5 | rs 662799 | Promoter | C | European |
| | | | Variant | | |
| (Sekar Kathiresan & Deepak | CDKN2A | rs10757274 | Intergenic | G | European |
| Srivastava, 2012) | | | | | |
| (Casas et al., 2006) | CETP | rs708272 | Intronic | C | European |
| (Sekar Kathiresan & Deepak | CXCL12 | rs1746048 | Intergenic | G | European |
| Srivastava, 2012); (Samani et | | | | | |
| al., 2007) | | | | | |
| (Harrison et al., 2012) | DAB21P | rs7025486 | Intergenic | A | European |
| (Casas et al., 2006) | LPL | rs328 | S447X | C | European |
| (Sekar Kathiresan & Deepak | MIA3 | rs17465637 | Intergenic | C | European |
| Srivastava, 2012) | | | | | |
| (Erdmann et al., 2009) | MRAS | rs9818870 | Intergenic | T | European |
| (Samani et al., 2007) | SMAD3 | rs17228212 | Intergenic | | European |
| (Sekar Kathiresan & Deepak | SORT1 | rs64677 | Intergenic | A | European |
| Srivastava, 2012) | | | | | |
| (Clarke et al., 2009) | LPA3 | rs3798220 | I1891M | C | European |
| (Clarke et al., 2009) | LPA10 | rs10455872 | Intergenic | C | European |
| (Casas et al., 2006) | ACE | rs4341 | Intergenic | G | European |
| (Casas et al., 2006) | APOB | rs1042031 | E4181K | A | European |
| (Sagoo et al., 2008) | LPL18 | rs1801177 | D9N | A | European, |
| | | | | | Mexican, Turks |

Source: (Beaney et al., 2015)

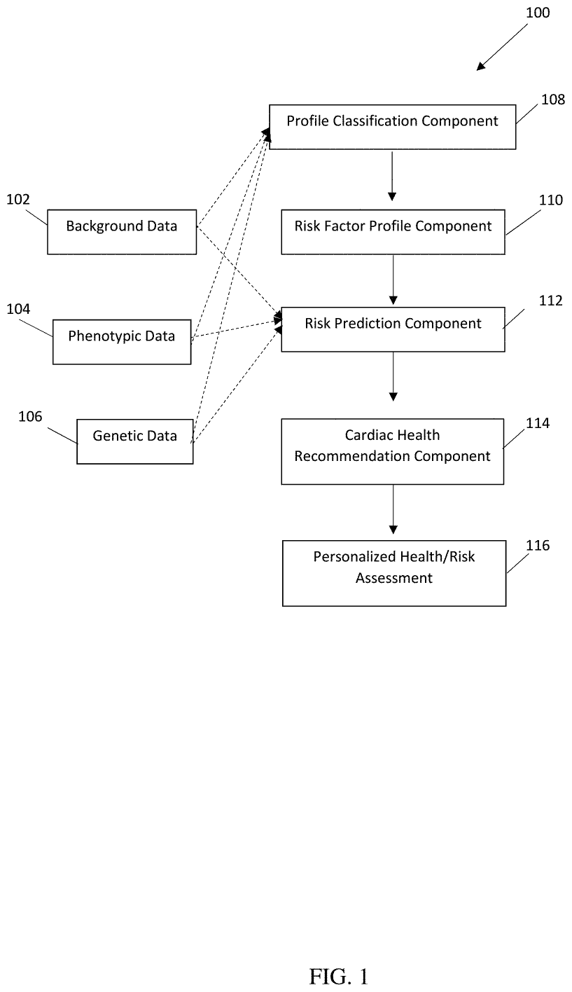

In some embodiments, the present invention provides processes, systems, and methods for providing a personalized cardiovascular disease (CVD) risk assessment for an individual based on the individual's background, phenotypic measurement data, and genetic information. FIG. 1 provides an overview 100 of exemplary systems and methods for deriving a CVD risk assessment according to the present invention. The process comprises obtaining a plurality of specific background data associated with the individual as shown at 102. The background data may include, but is not limited to sociodemographic data, personal and family medical history, medication use, diet and physical activity data, behavioral data, and any other pertinent lifestyle data.

The process further comprises obtaining phenotypic data associated with the individual as shown at 104. The phenotypic data may include, but is not limited to, biomedical or health care records, bioassays, blood and/or metabolic test data, medical imaging data, physiologic data, and the like, and combinations thereof. The method also includes obtaining genetic data associated with the individual as shown at 106. The genetic data may include, but is not limited to, genotypes, structural variations, sequences, and the like.

In accordance with the present disclosure, at least a portion of the background data, the phenotypic data, and/or the genetic data obtained from the individual is used in the determination of the individual's cardiovascular risk assessment. As an example, certain background data, such as personal medical history, diet and physical activity data, or behavioral data, may serve as prediction risk factors with respect to an individual's risk assessment. In one embodiment, one or more risk factors are selected to be included in the risk determination process.

In one embodiment, a profile classification component 108 analyzes background data associated with the individual to determine a profile classification for the individual. In a preferred embodiment, the profile classification component 108 analyzes at least a portion of the background information and assigns to at least one profile classification to the individual for determining CVD risk assessment. For example, based on an individual's ethnic background, the individual may be assigned to a profile classification associated with individuals of the same or similar ethnic background. As another example, based on an individual's sociodemographic data and severity of cardiovascular disease, the individual may be assigned to a profile classification associated with similarly situated individuals having the same or similar degree of cardiovascular symptoms.

In another embodiment, the profile classification component 108 may analyze at least a portion of the phenotypic information and/or the genetic information associated with the individual to determine a profile classification for the individual. For example, based on an individual's blood lipid levels and a genetic predisposition to cardiovascular disease, the individual may be assigned to a profile classification of individuals have similar blood test results and genetic data.

The profile classification assigned to an individual is then suitably used to determine the appropriate risk factors to be used in determining the individual's cardiovascular disease risk assessment. In one embodiment, the profile classification data is analyzed by a risk factor profile component 110 to determine risk factors and associated risk weights to be used in the risk assessment. Based on the specific profile classification assigned to the individual, the risk factor profile component 110 selects the appropriate risk factors to be included in the risk assessment. The risk factor profile component 110 also determines a risk weight to be assigned to each selected risk factor, generating a risk factor profile for the individual.

At least a portion of the background data, phenotypic data, and/or genetic data associated with the individual is subjected to a risk prediction algorithm or risk prediction component 112 to generate a personalized cardiovascular disease risk assessment for the individual. In one embodiment, based on the risk factor profile for the individual, data associated with each of the selected risk factors is integrated and subjected to the risk prediction component 112 to generate the personalized risk assessment. For example, in one embodiment, based on an individual's risk factor profile, the individual's risk assessment may be based, in part, on the individual's age, gender, marital status, cholesterol data, smoking status, and family history. The assigned risk weights for each risk factor would also be used in the determination. In another embodiment, based on another individual's risk profile, the risk prediction may be based, in part, on the individual's age, gender, education level, and blood pressure data. The risk prediction component 112 combines selected background data, phenotypic data, and genetic data into one uniform measurement of the individual's risk of developing cardiovascular disease.

In one embodiment, the risk prediction component 112 may also determine whether a particular factor increases or decreases the individual's chances of developing cardiovascular disease. Thus, the risk prediction component 112 may determine whether a particular factor is a risk factor or a protective factor for the individual.

In one embodiment, a cardiac health recommendation component 114 provides one or more personalized cardiac health recommendations 116 based on the personalized cardiovascular disease risk assessment. In one embodiment, the cardiac health recommendation component 114, based on a risk assessment indicating a very high likelihood of cardiac disease, might recommend immediate medical intervention. For example, high risk individuals may be subjected to at least one of the following: (1) secondary prevention guidelines to manage risk factors; (2) further evaluation, such as an exercise test, imaging, and/or angiography; (3) treatment of all major risk factors pharmacologically; and (4) appropriate lifestyle counseling.

In another embodiment, the cardiac health recommendation component 114, based on a moderate risk assessment, might recommend modifications to the individual's diet and exercise habits. As an example, a moderate risk individual may be subjected to at least one of the following: (1) lifestyle counseling; (2) pharmacologic treatment or one or more of the major risk factors; (3) initial follow-up in 3-6 months, annually thereafter; and (4) an exercise test.

In yet another embodiment, the cardiac health recommendation component 114, based on a low risk assessment, might recommend a continued health regime. For example, a low risk individual may be advised as to at least one of the following: (1) an appropriate lifestyle change; (2) re-evaluation in 3-12 months, with an additional evaluation in 3-5 years; and (3) reassurance as to the benefits of continuing to be low risk.

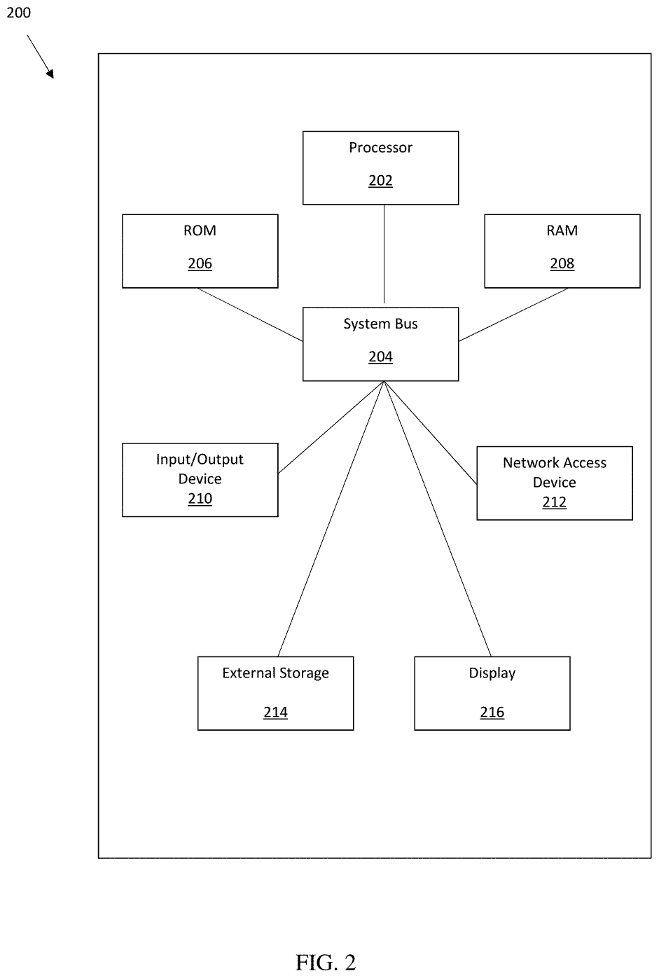

FIG. 2 is a high-level block diagram illustrating an example system environment for deriving personalized cardiovascular disease risk assessments according to the present disclosure. The system 200 is shown as a hardware device, but may be implemented in various forms of hardware, software, firmware, special purpose processors, or a combination thereof. Some embodiments are implemented in software as a program tangibly embodied on a program storage device. By implementing with a system or program, semi-automated or automated workflows are provided to assist a user in generating personalized health assessments.

The system 200 is a computer, personal computer, server, PACs workstation, mobile computing device, imaging system, medical system, network processor, network, or other now know or later developed processing system. The system 200 includes at least one processor 202 operatively coupled to other components via a system bus 204. The processor 202 may be, or may comprise, any suitable microprocessor or microcontroller, for example, a low-power application-specific controller (ASIC) and/or a field programmable gate array (FPGA) designed or programmed specifically for the task of controlling a device as described herein, or a general purpose central processing unit (CPU). In one embodiment, the processor 202 may be implemented on a computer platform, wherein the computer platform includes an operating system and microinstruction code. The various processes, methods, acts, and functions described herein may be either part of the microinstruction code or part of a program (or combination thereof) which is executed via the operating system as discussed below.

The other components include memories (ROM 206 and/or RAM 208), a network access device 212, an external storage 214, an input/output device 210, and a display 216. Furthermore, the system 200 may include different or additional entities.

The input/output device 210, network access device 212, or external storage 214 may operate as an input operable to receive at least a portion of at least one of the genotypic information and the phenotypic measurements. Input may be received from a user or another device and/or output may be provided to a user or another device via the input/output device 210. The input/output device 210 may comprise any combinations of input and/or output devices such as buttons, knobs, keyboards, touchscreens, displays, light-emitting elements, a speaker, and/or the like. In an embodiment, the input/output device 210 may comprise an interface port (not shown) such as a wired interface, for example a serial port, a Universal Serial Bus (USB) port, an Ethernet port, or other suitable wired connection. The input/output device 210 may comprise a wireless interface (not shown), for example a transceiver using any suitable wireless protocol, for example Wi-Fi (IEEE 802.11), Bluetooth®, infrared, or other wireless standard. In an embodiment, the input/output device 210 may comprise a user interface. The user interface may comprise at least one of lighted signal lights, gauges, boxes, forms, check marks, avatars, visual images, graphic designs, lists, active calibrations or calculations, 2D interactive fractal designs, 3D fractal designs, 2D and/or 3D representations, and other interface system functions.

The network access device 212 allows the computing system 200 to be coupled to one or more remote devices (not shown) such as via an access point (not shown) of a wireless network, local area network, or other coupling to a wide area network, such as the Internet. In that regard, the processor 202 may be configured to share data with the one or remote devices via the network access device 212. The shared data may comprise, for example, genetic information, phenotypic information, genetic risk prediction data, and the like. In various exemplary embodiments, the network access device 212 may include any device suitable to transmit information to and from another device, such as a universal asynchronous receiver/transmitter (UART), a parallel digital interface, a software interface or any combination of known or later developed software and hardware. The network access device 212 provides a data interface operable to receive at least a portion of at least one of the genotypic information and the phenotypic measurements.

The processor 202 has any suitable architecture, such as a general processor, central processing unit, digital signal processor, application specific integrated circuit, field programmable gate array, digital circuit, analog circuit, combinations thereof, or any other now known or later developed device for processing data. The processor 202 may be a single device or include multiple devices in a distributed arrangement for parallel and/or serial processing. Likewise, processing strategies may include multiprocessing, multitasking, parallel processing, and the like. A program may be uploaded to, and executed by, the processor 202.

The processor 202 performs the workflows, data manipulation of the genetic information, integration of phenotypic measurements with the genotypic information and/or other processes described herein. The processor 202 operates pursuant to instructions. The genotypic information and the phenotypic measurements may be stored in a computer readable memory, such as the external storage 214, ROM 206, and/or RAM 208. The instructions for implementing the processes, methods and/or techniques discussed herein are provided on computer-readable storage media or memories, such as a cache, buffer, RAM, removable media, hard drive or other suitable data storage media. Computer readable storage media include various types of volatile and nonvolatile storage media. The functions, acts or tasks illustrated in the figures or described herein are executed in response to one or more sets of instructions stored in or on computer readable storage media. The functions, acts or tasks are independent of the particular type of instructions set, storage media, processor or processing strategy and may be performed by software, hardware, integrated circuits, firmware, micro code and the like, operating alone or in combination. In one embodiment, the instructions are stored on a removable media device for reading by local or remote systems. In other embodiments, the instructions are stored in a remote location for transfer through a computer network or over telephone lines. In yet other embodiments, the instructions are stored within a given computer, CPU, GPU or system. Because some of the constituent system components and method acts depicted in the accompanying figures may be implemented in software, the actual connections between the system components (or the process steps) may differ depending upon the manner of programming.

The external storage 214 may be implemented using a database management system (DBMS) managed by the processor 202 and residing on a memory, such as a hard disk, RAM, or removable media. Alternatively, the storage 214 is internal to the processor 202 (e.g. cache). The external storage 214 may be implemented on one or more additional computer systems. For example, the external storage 214 may include a data warehouse system residing on a separate computer system, a PACS system, or any other now known or later developed storage system.

### II. Experimental

The following examples are provided in order to demonstrate and further illustrate certain preferred embodiments and aspects of the present disclosure and are not to be construed as limiting the scope thereof.

Ethics Statement

The relevant institutional review boards or ethics committees approved the research protocol used in the current analysis and all human participants gave written informed consent.

### A. Methodology

1. Participants and Samples

From Trinidad and Tobago, 778 participants were sampled, of which 252 were confirmed CVD diagnosed participants. For the purpose this analysis, CVD was defined as any disorder of the heart or blood vessels and included acute myocardial infarction (MI), silent MI, atherosclerosis, stents, coronary surgery, dyslipidemia and/or strokes. Data was collected on personal demographics, biometric measurements, medical history, family medical history, and lifestyle. Participants were also screened for 19 SNPs that are associated with myocardial infarction (MI), coronary heart disease (CHD), or CVD risk factors.

Measurements for weight, height, waist and hip circumference, blood pressure, lipid profiles were recorded for each participant. Where lipid profiles, blood pressure, weight, height, waist and hip circumference for participants were unknown, measurements were made using point of care devices (POCD). Lipid profiles which included total cholesterol (100 to 240 mg/dL), LDL (calculated), HDL (5-100 mg/dL) and Triglycerides (5-400 mg/dL)) was determined using the POC Cardiochek PA analyser (PTS Diagnostics, USA). The CardioChek PA analyser gave a calculated LDL value based on (Friedewald, Levy, & Fredrickson, 1972) formula. For instances where the participant's measurement value was outside the range of the point of care device (POCD), the respective minimum or maximum value of the POCD was utilised for analysis of data. Blood pressure was determined using an Omron blood pressure meter. Height was measured using a stadiometre to the nearest centimetre, whilst body weight was measured on a calibrated scale to the nearest gram. BMI was calculated on the basis of height and weight (BMI=weight (kg)/height² (m²)). The waist-hip circumference was measured according to the methods outlined by the consultation of WHO Experts (Consultation, 2008) using a tailor's measuring tape. Data from the biometric test performed were duplicated onto respective data collection sheets for Non-CVD and CVD participants

Buccal cavity swabs to provide DNA to screen for 19 Single Nucleotide Polymorphisms were also obtained from each participant. DNA was isolated using Cell Project DDK-50 isolation kit and protocol to produce an average yield of 1 to 10 g DNA (5 to 70 ng/μl) from an adult. Gel electrophoresis was performed on samples to ensure DNA was present. DNA was quantified using the Thermo scientific NanoDrop 2000/2000c unit at the Department of Microbiology, University of the West Indies, St. Augustine Campus. The Nanodrop was re-calibrated every 20 samples using the DNA storage solvent from Cell Projects. All samples were standardised to 5 ng/μl in Eppendorf tubes. Tubes were packaged and kept on dry ice for storage to the Centre for Cardiovascular Genetics, British Heart Foundation Laboratories, Institute of Cardiovascular Science, University College, London, University Street, London, United Kingdom.

DNA samples were then transferred to 96 well arrays at a volume of 120 μl at 1.25 ng/μl DNA per well (see Appendix 8 for the layout of arrays 1-9). The 96-well arrays of DNA samples were pipetted using the Biomek FX robot (Beckman Coulter) into three 384-well arrays, leaving 12 wells for No Template Controls (NTC) for each 384-well array (Error! Reference source not found.), at Genome Centre, Barts and the London, John Vane Science Centre, London (see Appendix 7—for layout of 5 384 arrays). The 384-well plates were placed in sterile bags and allowed to air dry overnight.

2. Single Nucleotide Polymorphism Selection

Nineteen SNPs were screened using either the TaqMan protocol or the KASPar protocol (Error! Reference source not found.). Nineteen single-nucleotide polymorphisms (SNPs) were chosen from 16 different genes for genotyping. All SNPs used were identified from the GWAS and candidate gene studies (Casas et al., 2006), 13 of which were identified from the most recent CARDIoGRAMplusC4D consortium (CARDIoGRAMplusC4D et al., 2013). Genotyping protocol utilised was determined based on the availability of resources as set forth in Table 3.

TABLE 3

| GENE | SNP | PROTOCOL |
|---|---|---|
| APOE112 | rs429358 | TaqMan |
| APOE158 | rs7412 | KASP |
| PCSK9 | rs11591147 | KASP |
| eNOS | rs1799983 | TaqMan |
| APOA5 | rs 662799 | KASP |
| CDKN2A | rs10757274 | KASP |
| CETP | rs708272 | KASP |
| CXCL12 | rs1746048 | KASP |
| DAB21P | rs7025486 | KASP |
| LPL | rs328 | KASP |
| MIA3 | rs17465637 | KASP |
| MRAS | rs9818870 | KASP |
| SMAD3 | rs17228212 | TaqMan |
| SORT1 | rs64677 | TaqMan |
| LPA (I1891M | rs3798220 | TaqMan |
| LPA10 | rs10455872 | TaqMan |
| ACE | rs4341 | KASP |
| APOB | rs1042031 | TaqMan |
| LPL (D9N | rs1801177 | TaqMan |

3. Genotyping

Genotyping was performed using published TaqMan (Applied Biosciences/Life Technologies, Grand Island, N.Y.), and KASPar (LGC Genomics, Herts, United Kingdom) technologies. 19 SNPs were selected that were associated with Coronary Heart Disease in genome-wide association studies (GWASs) or meta-analysis of candidate gene association studies was selected (CARDIoGRAMplusC4D et al., 2013; Casas et al., 2006).

Eight SNPs were genotyped using the TaqMan protocol. Polymerase chain reaction (PCR) reactions contained 6.25 ng DNA plated into a 384-well plate and dried down at room temperature overnight. PCR was performed in 1.9 μL volumes on a Bio-Rad 1000 thermal cycler (384 wells). Reactions contained 1.068 μL Kappa PCR Master Mix, 1.014 μL Sigma water and 0.053 μL of ×40 SNP. The 384-well plates were pressure sealed with Taqman optical covers and centrifuged for 30 s to ensure all contents were at the bottom of the well. The primer pool was amplified as follows: 90° C. for 2 min; 40 cycles of 95° C. for 10 min, 95° C. for 15 s; 60° C. for 1 min. After PCR plates were read immediately on the Taqman 7900HT detector (Applied Biosystems/Life Technologies) and then wrapped in foil and stored at 4° C. until results were validated.

Eleven SNPs were genotyped using the KASPar protocol (LGC Genomics, United Kingdom). Polymerase chain reaction (PCR) reactions contained 6.25 ng DNA plated into a 384-well plate and dried down at room temperature overnight. PCR was performed in 3.5 ul volumes on a Biorad 1000 thermal cycler (384 wells). Reactions contained 1.95 μL Kaspar PCR Master Mix, 1.95 μl Sigma water and 0.054 μl of ×40 SNP. The primer pool was amplified as follows: 94° C. for 15 min; 10 cycles of 94° C. for 20 s, 61-55° C. for 60 s (drop 0.6° C. per cycle); 26 cycles of 94° C. for 20 s, 55° C. for 60 s. For instances where genotyped clusters were not obtained after the initial KASP thermal cycle, the plate was read for up to additional three cycles. Further thermal cycling and plate reading was performed until defined genotype clusters were attained.

PCR reactions were read on the Taqman 7900HT detector (Applied Biosystems/Life Technologies) and then wrapped in foil and stored at 4° C. until results were validated. Error! Reference source not found. shows the list of SNPs genotyped using KASP protocol and the number of cycles required for adequate differentiation of genotyped clusters.

The manual calling option in the allelic discrimination application SDS software version 4 was then used to assign genotypes using the TaqMan 7900HT detector (Applied Biosystems/Life Technologies). Genotyping was cross-validated by in-house experts at the UCL Cardiovascular Genetics Laboratories.

### B. Cardiovascular Disease Risk Models

The risk models according to the present disclosure were developed using three different approaches—CHAID, Logistic Regression and Discriminant Analysis. It should be noted that these are classification and not future predictive models, so they are cross-sectional designs which use Chi-square tests (CHAID), odds ratios (Logistic regression) and discriminating group means (Discriminant Analysis to estimate significant classifying variables. CHAID analysis returns significant variables but does not yield a model equation, whereas Logistic Regression and Discriminant Analysis both give functions which can result in a score for each member of the sample. All give classification percentage of correctly classified and misclassified members for each of the CVD or Non-CVD groups. These models were compared to the performance of the three established CVD risk models viz. ASSIGN, Framingham and QRISK2 CVD (CVD) risk prediction models in discriminating between CVD and Non-CVD individuals.

1. Established Risk Models

The data collected from the participants was used to calculate risk scores from the three established CVD risk prediction models, QRISK2, Framingham, and ASSIGN. All three models determine the percentage risk of having a CVD event in the next 10 years. Theoretically, the scores for each model can range between 0% and 100%. According to the designers, individuals with scores of less than 10%, 10-20%, and greater than 20% are considered to have low, intermediate, and high risk, respectively of having a CVD event in the next 10 years. The range of scores for the participants are partitioned into three regions, Non-CVD, Non-Differentiated, and CVD for each standard risk model, using visual inspection of histograms of the predicted scores. Once it was established in which region each member falls, then the classification success (and misclassification error) was estimated for each Risk Model.

The percentage distributions of low, moderate, and high risk, predicted for the CVD (diagnosed CVD event) and Non-CVD (no diagnosed CVD event) groups by the three models, are given in Table 4. The results show that for persons who have had a CVD event, the established risk models identified 33% to 50% who are at high risk for developing another CVD event in the next 10 years while 24% to 31%%, are at moderate risk. The QRISK2 and Framingham model classified 21% and 22% of the CVD group as low risk. Analysis of variance among the means for each risk model showed significantly (p<0.001) higher mean scores for the CVD group compared to the Non-CVD group. (See Table 5).

TABLE 4
Percentage distribution of persons categorized by three established
Risk Models into different risk levels for Non-CVD and CVD groups

| | | Low Risk | Moderate Risk | High Risk |
|---|---|---|---|---|
| Group | Risk Model | <10% | 10-20% | >20% |
| Non-CVD | Framingham | 87 | 8 | 5 |
| | ASSIGN | 90 | 6 | 4 |
| | QRISK2 | 92 | 5 | 3 |
| CVD | Framingham | 22 | 28 | 50 |
| | ASSIGN | 43 | 24 | 33 |
| | QRISK2 | 21 | 31 | 48 |

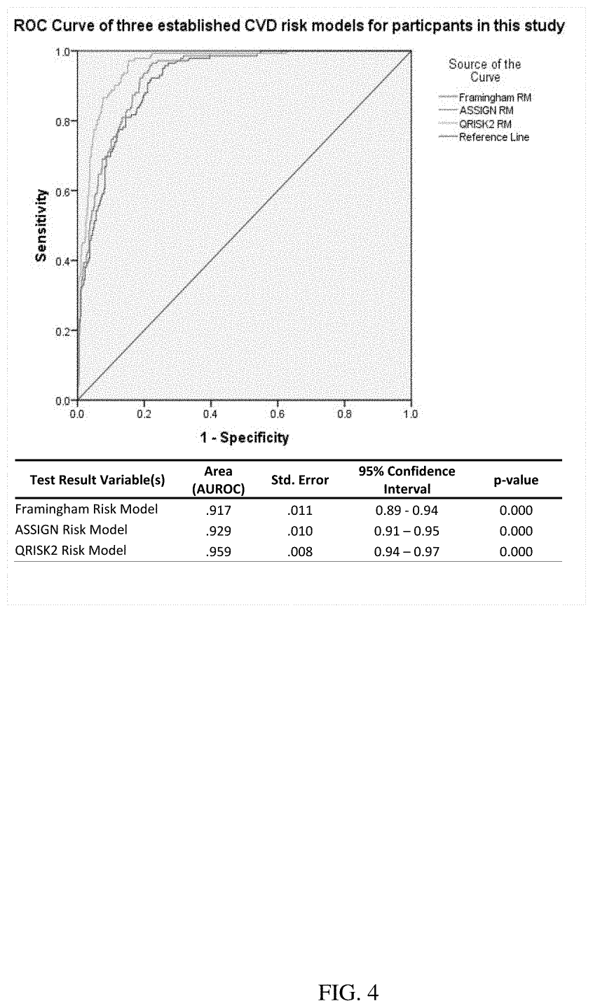

The scores from the three models are found to be significantly different (ANOVA p-value<0.001) for both the Non-CVD and CVD groups (Table 5). An assessment of their predictive ability, however, is obtained via the AUROC procedure. Receiver Operating Characteristic (ROC) curves were constructed (using SPSS V.22) for the scores from each risk model as shown in FIG. 4. This analysis yields a measure of predictive accuracy from the area under the curve (AUROC). The QRISK2 risk prediction model (AUROC=0.96) performed the best at predicting 10-year CVD risk among the study population compared to the ASSIGN (AUROC=0.93) and the Framingham risk prediction model (AUROC=0.92).

TABLE 5
Analysis of variance of means between Non-CVD and CVD participation
for the mean risk scores determined by the sample population

| | NON-CVD | | | CVD | | | |
|---|---|---|---|---|---|---|---|
| Risk Model | N | MEAN (SD) | | N | MEAN (SD) | | p-value |
| QRISK2 Risk | 526 | 2.7 | (6.6) | 252 | 23.5 | (17.4) | 0.000 |
| score | | | | | | | |
| ASSIGN Risk | 526 | 3.7 | (6.7) | 252 | 16.6 | (14.9) | 0.000 |
| Score | | | | | | | |
| Framingham | 485 | 3.9 | (7.2) | 142 | 22.8 | (14.8) | 0.000 |
| Risk Score | | | | | | | |

Whilst the three approaches to predicting CVD risk are able to successfully discriminate between the CVD participants and the Non-CVD participants. From the literature, all three methods have been reported to correctly predict less than 70% of cases, with a high ratio of false positive predictions to true predictions. Bearing this in mind, analysis was performed with respect to risk scores for Framingham, ASSIGN, and QRISK2 models, to quantify the amount of CVD persons being grouped as Non-CVD. From this analysis, three ranges of scores were deduced, the participants that were clearly Non-CVD, those that were clearly CVD, and then those participants that had a mix of both Non-CVD and CVD, termed the Non-Differentiating range (see Table 6). Sensitivity measures the proportion of CVD participants correctly identified and specificity measures the proportion of Non-CVD participants correctly identified, were also determined for the three established risk models.

TABLE 6
Table of ranges, specificity and sensitivity for Non-CVD, CVD and Non-Differentiating
for ASSIGN, Framingham and QRISK2 risk models

| | | | | | NON- | |
|---|---|---|---|---|---|---|
| | CVD | | DIFFERENTIATING | | NON-CVD | |
| RISK MODEL | Range | Sensitivity | Range | % | Range | Specificity |
| ASSIGN RS | 0.03 to | 0.49 | 5.00 to 12.50 | 16.8 | 12.51 to | 0.81 |
| | 4.99 | | | | 69.39 | |
| Framingham RS | 0.00 to | 0.45 | 10.00 to 22.50 | 14 | 22.51 to | 0.88 |
| | 9.99 | | | | 65.10 | |
| QRISK2 RS | 0.00 to | 0.62 | 5.00 to 15.00 | 13.5 | 15.01 to | 0.87 |
| | 4.99 | | | | 81.04 | |

QRISK2 had the best sensitivity and specificity values (0.62 and 0.87, respectively) while Framingham had the lowest sensitivity (0.45) and ASSIGN had the lowest specificity (0.81). The ASSIGN model had the largest percentage of persons (16.8%) classified in the Non-Differentiating range followed by the Framingham (14%) and QRISK2 (13.5%) model. This indicates that the QRISK2 model would be best at distinguishing between Non-CVD and CVD patients, with the smallest number of persons being classified as Non-Differentiating. These results corroborate what was seen with the AUROC.

2. Personalized Risk Models

Chi-Square Automatic Interaction Detector (CHAID) Trees

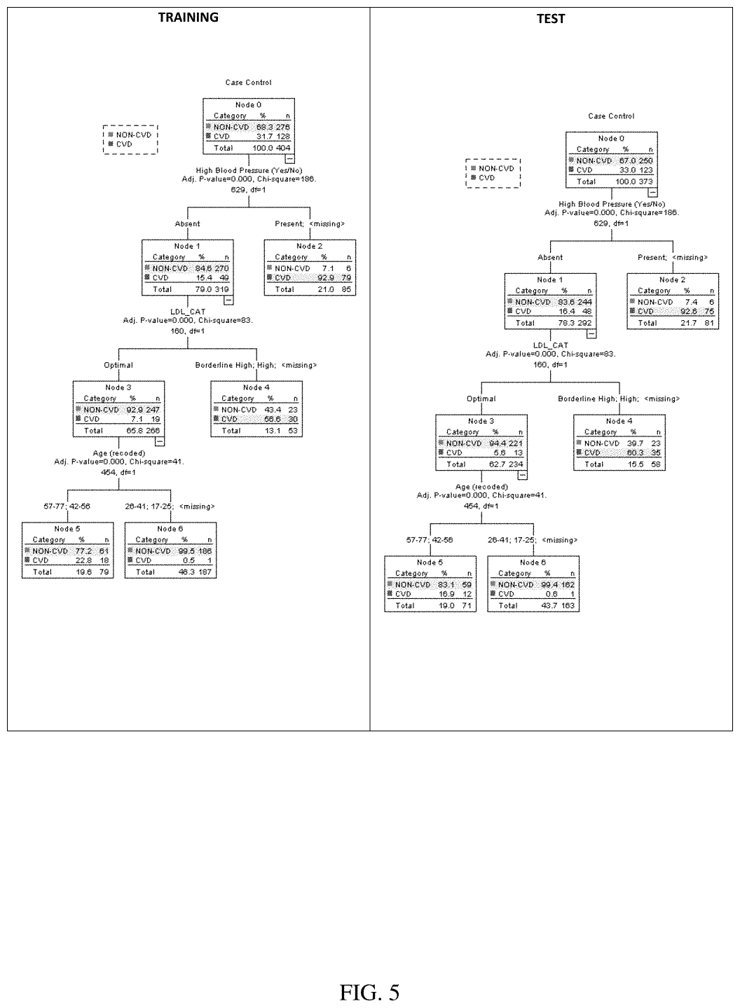

Chi-square Automatic Interaction Detector (CHAID) analysis was performed to identify the most important categorical predictors that were able to discriminate between Non-CVD and CVD. Exhaustive CHAID, a modification to the basic CHAID algorithm, performs a more thorough merging and testing of predictor variables. The decision tree algorithm (CHAID) cross-validated the sample model by randomly splitting the sample into two, where one set of participants (Training) were used to develop the model and another set of participants were used to test the model (Test). The CHAID was then performed three times (3 models) to partition the data into statistically significant subgroups that were mutually exclusive and exhaustive. The results showed that age and the presence of High Blood Pressure were consistent predictors for all three models (see Table 8). Model 3 was the most successful at predicting the CVD group in the sample at 85% in the Training sample and 88% in the Test sample compared to the Model 1 and 2 (see Table 7). Models 2 and 3 had the same three predicators but with different classification prediction, which is due to the randomized splitting of the sample into Training and Test. Three major predictor variables reached significance to be included in model 3, namely the presence of high blood pressure, age in years and LDL levels. This model had an overall classification accuracy of 88.4% with its ability to discriminate between Non-CVD and CVD participants. The tree analysis for Model 3 in FIG. 5 shows the 3-level CHAID tree with a total of 8 nodes, of which 5 were terminal nodes. All individuals were divided into 2 subgroups from the root node to leaf nodes through different branches. The likelihood of having a CVD varied from 0 to 32%. For example, 93% of persons with High Blood Pressure was likely to have a CVD. By using this type of decision tree model, researchers can identify the combinations of factors that constitute the highest (or lowest) risk for a condition of interest.

TABLE 7
CHAID split sample for the three models with percentage
correctly predicted for the CVD group and mean
overall correctly predicted percentage.

% CORRECT PREDICTED FOR CVD GROUP

| | | | MEAN OVERALL |
|---|---|---|---|
| | TRAINING | TEST | (NON-CVD AND CVD) |
| MODEL 1 | 59.3 | 60.9 | 85.8 |
| MODEL 2 | 69.0 | 54.8 | 85.9 |
| MODEL 3 | 85.2 | 89.4 | 88.4 |

TABLE 8
Summary of the three CHAID tress developed

| | MODEL 1 | MODEL 2 | MODEL 3 |
|---|---|---|---|
| Specifications | | | |
| Growing Method | EXHAUSTIVE CHAID | EXHAUSTIVE CHAID | EXHAUSTIVE CHAID |
| Dependent Variable | Case Control | Case Control | Case Control |
| Independent | Age, Sex, Ethnicity, | Age, Sex, Ethnicity, | Age, Sex, Ethnicity, |
| Variables | Marital_status, Highest_Edu, | Marital_status, | Marital_status, Highest_Edu, |
| | Smoking, BMI, Tot. Chol, | Highest_Edu, Smoking, | Smoking, BMI, Tot. Chol, |
| | Triglycerides, LDL, HDL, | BMI, Tot. Chol, | Triglycerides, LDL, HDL, |
| | TC_HDL, SBP, DBP, | Triglycerides, LDL, HDL, | TC_HDL, SBP, DBP, |
| | Diabetic, High_Cholesterol, | TC_HDL, SBP, DBP, | Diabetic, High_Cholesterol, |
| | Chronic_Kidney_Disease, | Diabetic, High_Cholesterol, | Chronic_Kidney_Disease, |
| | Atrial_fibrillation, | Chronic_Kidney_Disease, | Atrial_fibrillation, |
| | Rheumatoid_arthritis, HBP, | Atrial_fibrillation, | Rheumatoid_arthritis, HBP, |
| | Family_HBP, | Rheumatoid_arthritis, HBP, | Family_HBP, |
| | Family_High_Cholest., | Family_HBP, | Family_High_Cholest., |
| | Family_Diabetes | Family_High_Cholest., | Family_Diabetes |
| | Family_CVD | Family_Diabetes | Family_CVD |
| | | Family_CVD | |
| Validation | Split Sample | Split Sample | Split Sample |
| Maximum Tree | 3 | 3 | 3 |
| Depth | | | |
| Minimum Cases in | 100 | 100 | 100 |
| Parent Node | | | |
| Minimum Cases in | 50 | 50 | 50 |
| Child Node | | | |
| Results | | | |
| Independent | HBP, Age, Ethnicity | HBP, LDL, Age | HBP, LDL, Age |
| Variables Included | | | |
| Number of Nodes | 8 | 7 | 7 |
| Number of | 5 | 4 | 4 |
| Terminal Nodes | | | |
| Depth | 3 | 3 | 3 |

Logistic Regression

A forward LR binary logistic regression was performed in SPSS v.21 for the sample population on nineteen variables (See Table 9). The final logistic regression model obtained ten predictors to discriminate between the CVD and Non-CVD groups.

TABLE 9
Binary logistic regression analysis (forward LR) of the main contributing
parameters distinguishing between Non-CVD and CVD participants.

| | B | S.E. | Wald | df | Sig. | Exp (B) |
|---|---|---|---|---|---|---|
| Age (years) | 0.119 | 0.03 | 21.81 | 1 | .000 | 1.127 |
| Sex (Female) | −1.333 | 0.62 | 4.64 | 1 | .031 | .264 |
| Marital status | | | 7.50 | 2 | .023 | |
| Marital status (Married) | 1.502 | 0.71 | 4.43 | 1 | .035 | 4.490 |
| Marital status (Divorced/ | −0.148 | 0.86 | 0.03 | 1 | .863 | .862 |
| Separated/Widowed) | | | | | | |
| Smoking | | | 8.26 | 2 | .016 | |
| Smoking (Ex-Smoker) | 1.320 | 0.88 | 2.25 | 1 | .134 | 3.744 |
| Smoking (Smoker) | 2.619 | 0.99 | 6.99 | 1 | .008 | 13.725 |
| TC/HDL ratio | −0.5550 | 0.21 | 6.72 | 1 | .010 | .574 |
| LDL (mg/dL) | 0.026 | 0.01 | 9.90 | 1 | .002 | 1.027 |
| High cholesterol (Present) | 1.952 | 0.55 | 12.64 | 1 | .000 | 7.045 |
| Atrial fibrillation (Present) | 2.867 | 1.34 | 4.57 | 1 | .033 | 17.586 |
| High blood pressure | 4.272 | 0.78 | 30.00 | 1 | .000 | 71.635 |
| (Present) | | | | | | |
| History of family CVD | 1.957 | 0.54 | 13.14 | 1 | .000 | 7.077 |
| (Present) | | | | | | |
| Constant | −10.913 | 1.88 | 33.69 | 1 | .000 | .000 |

The results showed that the predictors included age (years), TC/HDL and LDL levels (mg/dL), female sex, smoking, marital status of divorced/separated/widowed, presence of high cholesterol, presence of atrial fibrillation, presence of high blood pressure and presence of a family history of CVD (Table 9). The model explained 55% of the variance (Cox and Snell R²=0.55). The most impactful factor was the presence of high blood pressure (B=4.3); followed by the presence of atrial fibrillation (B=2.9) and if the participant was a smoker (B=2.6). Ninety-nine percent (99%) of the Non-CVD participants were correctly predicted while 88% of the CVD participants were correctly predicted using this model.

In order to determine which CVD participants were being classified as Non-CVD and vice versa, LinRisk scores were computed using the following algorithm from the logistic regression. A histogram was plotted in order to establish the ranges that were clearly Non-CVD participants, clearly CVD participants and the range that had a mix of Non-CVD and CVD participants (i.e. Non-Differentiating range). LinRisk and was computed as follows:

$$LinRisk = - 10.913 + 0.119 ( Age in years ) - 1.333 ( Female ) + 1.502 ( Married ) + 2.619 ( Smoker ) - 0.555 ( TC \text{/} HDL in mg \text{/} dL ) + 0.026 ( LDL in mg \text{/} dL ) + 1.952 ( Presence of High Cholesterol ) + 2.867 ( Presence of Atrial Fibrillation ) + 4.272 ( Presence of High Blood Pressure ) + 1.957 {( Presence of Family CVD )}^{″}$$

Where, if the participant was female, married, a smoker, had a presence of high cholesterol, a presence of atrial fibrillation, a presence of high blood pressure or a presence of a family history of CVD, they were given a value of 1, and absence of all other categories were given a value of 0.

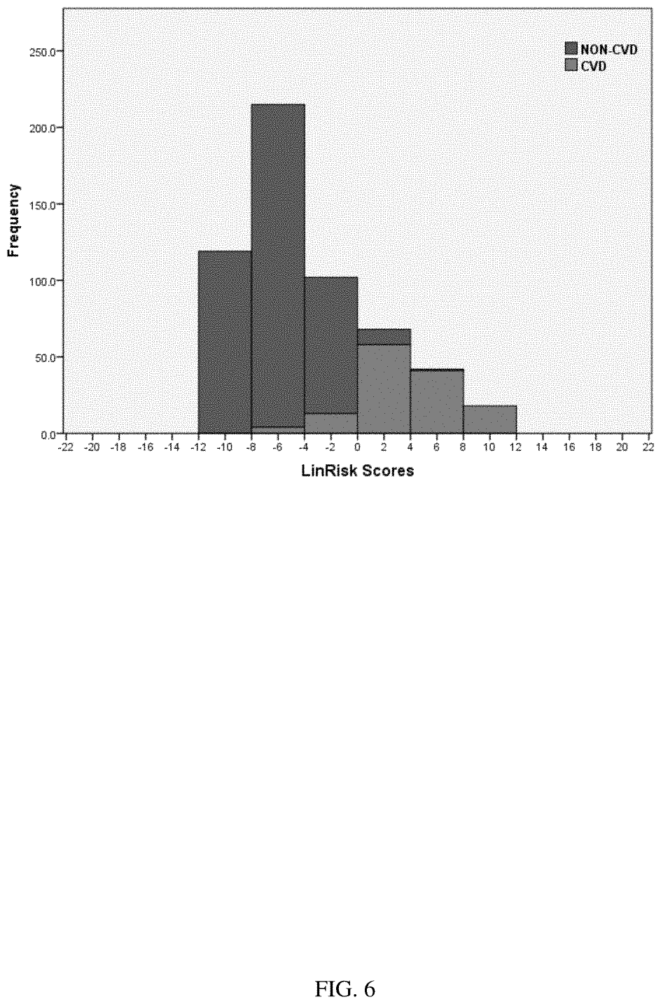

The ranges were defined as Clearly Non-CVD (−12.0 to −4.01)), Non-differentiating range (−4.00 to 0.00) and Clearly CVD (0.01 to 12.0) for the logistic regression. The Non-differentiating range included those CVD participants that were classified as a Non-CVD participant (See FIG. 6) according to this model. The results of the classification will be shown after the Discriminant Model is described so that their classification sensitivities and specificities can be compared.

Discriminant Analysis (DA)

Discriminant Analysis (DA) was performed in SPSS for the sample population on nineteen variables (See Table 2Error! Reference source not found.). The Discriminant analysis gave standardized and unstandardized canonical discriminant function coefficients for 13 significant variables (p<0.05), in order to predict those that belonged in the Non-CVD or CVD group (See Table 10Error! Reference source not found.).

Predictor variables were age (years), sex, highest education level, marital status, smoking status, ratio of TC/HDL (mg/dL), levels of Low Density Lipoprotein (LDL) (mg/dL), systolic and diastolic blood pressure levels (mmHg), presence of high cholesterol, presence of atrial fibrillation and the presence of a family history of CVD. Box's M was significant (p<0.000) with dissimilar log determinants (3.9 for the Non-CVD group and 7.3 for the CVD group) which indicates a violation of the assumption of equal variance-covariance matrices, leading to greater classification errors (specifically, DA will tend to classify cases in the group with the larger variability). However, given the large sample, this problem is not regarded as serious.

The discriminant function revealed a significant association between groups and all predictors. Ninety-six percent (96%) of the Non-CVD participants were correctly predicted while 95% of the CVD participants were correctly predicted using this model. The cross-validated classification showed that overall 95.7% were correctly classified.

TABLE 10
Standardized and unstandardized canonical discriminant function
coefficients for the 12 variables in the discriminant model
distinguishing Non-CVD and CVD participants for the sample.

| Variables | Unstandardized | Standardized |
|---|---|---|
| Age (years) | 0.025 | 0.323 |
| Sex (Male/Female) | −0.310 | −0.154 |
| Secondary Education Level | 0.432 | 0.126 |
| Tertiary Education Level | 0.564 | 0.262 |
| Married | 0.237 | 0.110 |
| Ex-Smoker | 1.379 | 0.278 |
| TC/HDL ratio (mg/dL) | −0.161 | −0.240 |
| LDL (mg/dL) | 0.008 | 0.298 |
| Systolic blood pressure (mmHg) | 0.009 | 0.192 |
| Diastolic blood pressure (mmHg) | −0.024 | −0.314 |
| High Cholesterol (Yes/No) | 0.889 | 0.282 |
| Atrial fibrillation (Yes/No) | 0.872 | 0.176 |
| High Blood Pressure (Yes/No) | 2.892 | 0.654 |
| Family history of CVD (Yes/No) | 0.488 | 0.206 |
| Constant | −1.432 | |

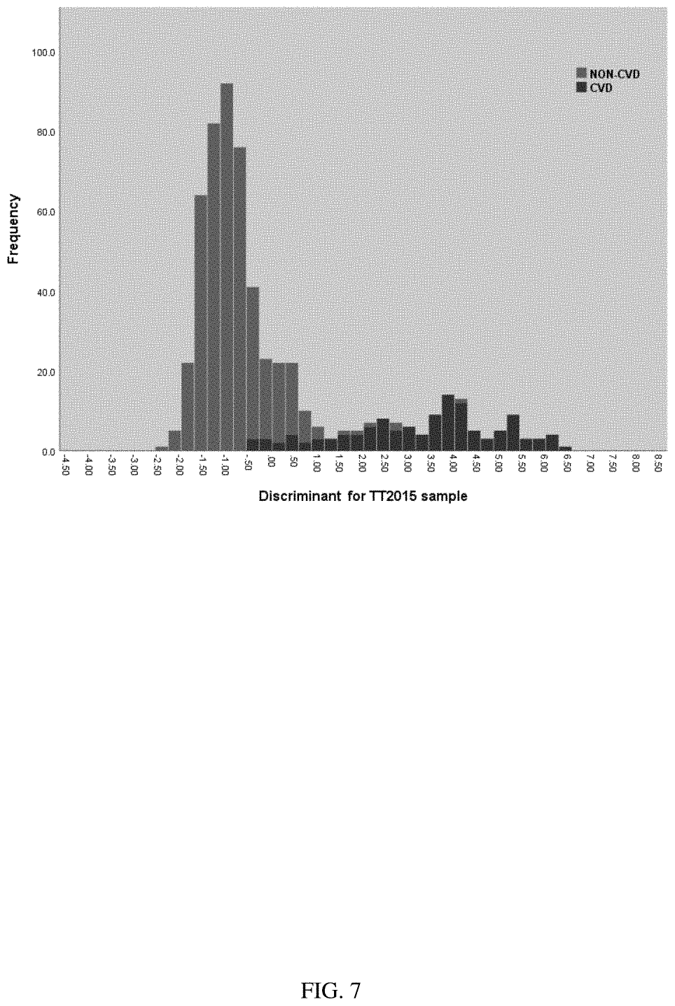

In order to determine which CVD participants were being classified as Non-CVD and vice versa, DiscrimRisk scores were determined from the analysis output. As shown in FIG. 7, a histogram was plotted in order to establish the ranges that were clearly Non-CVD participants, clearly CVD participants and the range that had a mix of Non-CVD and CVD participants (i.e. Non-Differentiating range). DiscrimRisk and was computed as follows:

$$DiscrimRisk = - 1.432 + 0.025 ( Age in years ) - 0.321 ( M = 0 , F = 1 ) + 0.432 ( Secondary Education Level = 1 ) + 0.564 ( Tertiary Education Level = 1 ) + 0.237 ( Married = 1 ) ++ 1.379 ( ExSmoker = 1 ; NonSmoker or Smoker = 0 ) - 0.161 ( \frac{TC}{HDL} ratio ) + 0.008 ( LDL in \frac{mg}{dL} ) + 0.009 ( Systolic BP in mmHg ) - 0.024 ( diastolic BP in mmHG ) + 0.889 ( presence of high cholesterol = 1 ) + 0.872 ( presence of atrial fibrillation = 1 ) + 2.892 ( presence of high blood pressure = 1 ) + 0.488 ( presence of a family history of CVD = 1 )$$

The ranges for the Discriminant model were defined as Clearly Non-CVD (−2.7 to −0.6), Non-differentiating range (−0.5 to 1.4) and Clearly CVD (1.5 to 6.6). The Non-differentiating range included CVD participants that were classified as Non-CVD participants and vice versa (See FIG. 7).

Cardiac Health Recommendation Protocol

In one embodiment as discussed above, the cardiac health recommendation component 114 provides one or more personalized cardiac health recommendations 116 based on the personalized cardiovascular disease risk assessment. In one embodiment, the cardiac health recommendation component 114, based on a risk assessment using either the Logistic Regression model or the Discriminant Analysis model, would recommend medical intervention according to the likelihood of cardiovascular disease. In one embodiment, the cardiac health recommendation protocol may vary based on the ethnicity of an individual and/or risk assessment model used to predict the risk of cardiovascular disease.

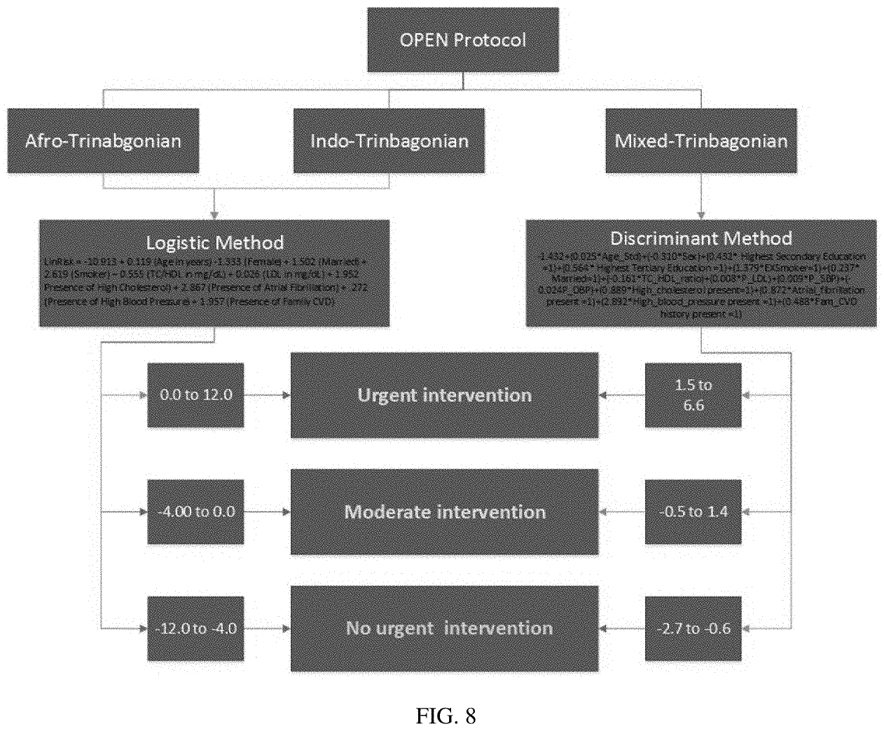

FIG. 8 provides one illustrative embodiment of a cardiac health recommendation protocol 800, wherein the urgency of medical intervention varies based on ethnicity of the individual and the risk assessment model used. The protocol 800 provides recommendations for medical intervention for three ethnicities, Afro-Trinbagonian 802, Indo-Trinbagonian 804, and Mixed-Trinbagonian 806, based on risk assessment scores from either the Logistic Method 808 or the Discriminant Method 810. As shown in FIG. 8, the Afro-Trinbagonian 802 and Indo-Trinbagonian 804 individuals were assessed using the Logistic Method 808 and Mixed-Trinbagonian 806 individuals were assessed using the Discriminant Method 810.

As illustrated in FIG. 8, the protocol 800 provides for three levels medical intervention, Urgent Intervention 812, Moderate Intervention 814, and No Urgent Intervention 816. Urgent intervention 812 is recommended for those individuals having a risk assessment score ranging from 0.0 to 12.0 as shown at 818 using the Logistic Method 808 or a risk assessment score ranging from 1.5 to 6.6 as shown at 820 using the Discriminant Method 810. Such individuals are typically defined as Clearly-CVD. Moderate intervention 814 is recommended for those individuals having a risk assessment score ranging from −4.0 to 0.0 as shown at 822 using the Logistic Method 808 or a risk assessment score ranging from −0.5 to 1.4 as shown at 824 using the Discriminant Method 810. Such individuals are typically defined as Non-Differentiating. No urgent intervention 816 is recommended for those individuals having a risk assessment score ranging from −12.0 to −4.0 as shown at 826 using the Logistic Method 808 or a risk assessment score ranging from −2.7 to −0.6 as shown at 828 using the Discriminant Method 810. Such individuals are typically defined as Clearly Non-CVD.

3. Summary of the Risk Models

Summary of Personalized Risk Prediction Models

The three models tested for discrimination of non-CVD and CVD participants in the TT2015 sample were the CHAID, Logistic regression and discriminant analysis. All three models (see Error! Reference source not found.e 11) recognised the three predictors from the CHAID viz. presence of High Blood Pressure (HBP), levels of Low-density Lipoprotein (mg/dL) and age (years); as predictors for CVD. It is interesting to note that Model 1, of the CHAID although not as successful as Models 2 and 3, recognised Ethnicity as an important predictor of CVD (Table 7). From the three models, the logistic regression (Non-CVD: 76% and CVD: 86%) and discriminant analysis (Non-CVD: 98% and CVD: 87%) were able to correctly classify Non-CVD and CVD participants compared to the CHAID model (Error! Reference source not found.able 7).

Comparison of Variables in all CVD Risk Prediction Models

The six models for deciding if a person has or will have a CVD were the ASSIGN, Framingham (2008), QRISK2, CHAID, Logistic Regression (LG) and Discriminant Analysis (DA). The first three models are established models that estimate the risk of a person having a CVD in the next 10 years (D'Agostino et al., 2008; Hippisley-Cox et al., 2007; Woodward, Brindle, & Tunstall-Pedoe, 2007). The latter three models were developed using statistical tools in SPSS to discriminate between CVD (case) and Non-CVD (control) participants.

TABLE 11
Variables used in the six CVD Risk Models

| | Logistic | | | | | |
|---|---|---|---|---|---|---|
| Predictor | regression | Discriminant | CHAID | Framingham | ASSIGN | QRISK2 |
| Age (years) | ✓ | ✓ | ✓ | ✓ | ✓ | ✓ |
| Presence of HBP | ✓ | ✓ | ✓ | | | ✓ |
| Family history of CVD | ✓ | ✓ | | ✓ | ✓ | ✓ |
| Presence of High Cholesterol | ✓ | ✓ | | | | |
| Presence of Atrial Fibrillation | ✓ | ✓ | | | | ✓ |
| Sex | ✓ | ✓ | | ✓ | ✓ | ✓ |
| Smoking | ✓ | ✓ | | ✓ | ✓ | ✓ |
| TC/HDL | ✓ | ✓ | | ✓ | ✓ | ✓ |
| Marital status | ✓ | ✓ | | | | |
| LDL | ✓ | ✓ | ✓ | | | |
| Diabetic | | | | ✓ | ✓ | ✓ |
| Diastolic BP | | ✓ | | | | ✓ |
| No. of cigarettes/day | | | | | ✓ | ✓ |
| Presence of Left Ventricular | | | | | ✓ | |
| Hypertrophy | | | | | | |
| Social deprivation^{†} | | | | | ✓ | ✓ |
| Systolic BP | | ✓ | | ✓ | ✓ | ✓ |
| BMI | | | | | | ✓ |
| Presence of Rheumatoid arthritis | | | | | | ✓ |
| Presence of chronic kidney disease | | | | | | ✓ |
| Ethnicity | | | | | | ✓ |
| Education Level | | ✓ | | | | |
| Total number of predictors in the | 10 | 13 | 3 | 7 | 10 | 14 |
| model | | | | | | |

QRISK2 used 14 predictors to estimate CVD risk, which was followed by the Discriminant Analysis (13 predictors), Framingham (10 predictors) and Logistic Regression (10 predictors), ASSIGN (7 predictors) and lastly CHAID with 3 predictors (Table 11). The number of predictors had no influence on the goodness of fit of the models.

Age was a common predictor for all six models. The presence of a family history of CVD, sex, smoking status and the ratio of total cholesterol to high-density lipoprotein (HDL), were predictors in all models except the CHAID.

All 10 predictors in the Logistic Regression were used in the Discriminant Analysis (Error! Reference source not found.). While a family history of CVD was the strongest predictor in the Logistic Regression, the presence of High Blood Pressure was the strongest predictor for the Discriminant Analysis model. Additional factors in the Discriminant Analysis model included diastolic BP levels, systolic BP levels, and highest education levels. Systolic BP is also predictors in the three established CVD risk prediction models, but diastolic BP is only included in the QRISK2 prediction model (Table 11).

The presence of Diabetes was a predictor for the three established CVD risk models but was not recognised as a predictor in the three models developed in this study. The inclusion of a social deprivation score as a predictor for ASSIGN and QRISK2 was not possible for any of the six models because it does not exist for the Trinidad and Tobago population. It is expected that the urban intensity index recently published for Trinidad and Tobago, will be evaluated for use as such a factor in the near future.

Classification for the 5 CVD Risk Prediction Models

The CVD risk scores produced by the QRISK2, Framingham and ASSIGN models gives the likelihood of having a CVD event in the next 10 years and was developed from cohort studies by evaluating a sample population over many decades. These models ideally should recognise if a participant has a CVD at present and not in 10 years. The nature of the CHAID model is unable to provide a risk score for each individual, and as such was not evaluated in the comparative analysis of the CVD risk models.

Sensitivity and specificity are terms used to evaluate a clinical test. In this study, these clinical tests are the 5 CVD risk prediction models. The sensitivity of a test refers to the ability of the test to correctly identify those participants with the disease while the specificity of a clinical test refers to the ability of the test to correctly identify those participants without the disease.

TABLE 12
Classification characteristics for 5 CVD Risk Prediction Models evaluated.

| CHARACTERISTIC | ASSIGN | FRAM.^{a} | QRISK2 | LR^{b} | DA^{c} |
|---|---|---|---|---|---|
| Sample Population | Scotland | USA^{d} | UK^{d} | T&T^{f} | T&T |
| Sensitivity | 49.6 | 44.7 | 62.1 | 86.0 | 94.7 |
| Specificity | 80.3 | 87.4 | 86.4 | 76.4 | 96.3 |
| Positive predictability | 77.9 | 77.8 | 87.0 | 91.4 | 86.4 |
| Negative predictability | 87.1 | 92.9 | 92.3 | 98.2 | 98.7 |
| Non-Differentiating | 5.00 to | 10.00 to | 5.00 to | −4.00 to | −0.5 to |
| risk score range | 12.50 | 22.50 | 15.00 | 0.00 | 1.4 |
| Total % population in | 16.84 | 14.04 | 13.50 | 18.2 | 20.2 |
| Non-differentiating range | | | | | |
| Relative Risk of CVD^{g} | 1.99 | 3.74 | 2.58 | 0.46 | 0.76 |
| being in non-differentiating range | | | | | |

^{a}Framingham (FRAM.)CVD risk model (2008)
^{b}Logistic regression (LR) CVD model
^{c}Discriminant analysis (DA) CVD model
^{d}United States of America
^{e}United Kingdom
^{f}Trinidad and Tobago
^{g}Cardiovascular Disease

Both tests are independent of the population of interest subjected to the test. Positive and negative predictive values are useful when considering the value of a test to a clinician. They are dependent on the prevalence of the disease in the population of interest. The positive predictability is the percentage of patients with a positive test who actually have the disease while negative predictability is the percentage of patients with a negative test who do not have the disease.

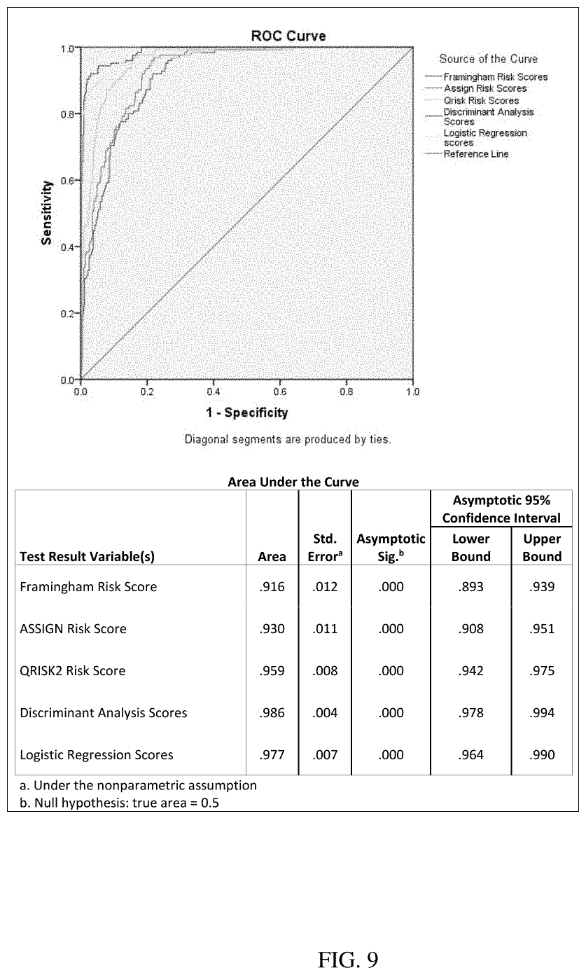

Receiver operator characteristic (ROC) curves are a plot of false positives against true positives for all cut-off values. The area under the curve of a perfect test is 1.0 and that of a useless test, no better than tossing a coin, is 0.5. ROC curves for 5 of the models were plotted (see FIG. 9) and the Discriminant Analysis performed the best (AUROC=0.986) while the Framingham risk model performed the poorest (AUROC=0.916). This indicates that the Discriminant Analysis was best at classifying both Non-CVD and CVD participants.

As shown in Table 12, the sensitivity and positive predictability for the Discriminant were 95% and 86%, respectively, for the logistic regression model. The results show that from the three established CVD risk models (i.e. ASSIGN, Framingham, and QRISK2), the QRISK2 model had the highest sensitivity (65%) and positive predictability (87%), which was still relatively low compared to the discriminant analysis and logistic regression. This means that 65% of the CVD participants in the QRISK2 model will be detected as having a CVD (true positives) and that 35% of the CVD participants will go undetected (false negatives). A high sensitivity is clearly important where the test is used to identify a serious but treatable disease such as a CVD.

The results show that the Discriminant Analysis and Logistic Regression models were statistically more robust compared to the ASSIGN, Framingham, and QRISK2.

Relative Risk of CVD in the Non-Differentiating Range

The Non-Differentiating zone is the range of scores in which there is a mix of CVD and Non-CVD participants. This was evaluated from histograms of risk scores for ASSIGN, Framingham, QRISK2, Logistic Regression and Discriminant Analysis. Each risk model had ranges that were clearly Non-CVD participants, clearly CVD participants and the Non-Differentiating range that had a mix of Non-CVD and CVD participants (see Table 12).

The Discriminant Analysis had the largest percentage of participants (20.2%), and the QRISK2 had the lowest percentage of participants (13.5%) in the Non-Differentiating range of the TT2015 sample population. The relative risk of a CVD participant being in this range was the highest for the three established risk models (1.99 to 3.74) and lowest for the logistic and discriminant models (0.46 and 0.59). This meant that if a participant received a QRISK2 score of 5.20 (the Non-Differentiating range) that there would be 2.6 times greater probability that this could be a CVD participant rather than a Non-CVD participant. This implies that the Non-Differentiating range was more likely to include CVD participants for the ASSIGN, Framingham, and QRISK2 compared to the Logistic Regression and Discriminant Analysis. The Logistic Regression and Discriminant Analysis were more likely to include Non-CVD participants in the Non-Differentiating ranges, which is a preferable to misclassifying CVD participants.

Example of Misclassification Using Established Risk Models

Participant X is a Trinidadian male, 40 years old, of mixed ethnicity, married, tertiary educated, has high cholesterol (TC=224, HDL=56, LDL=144, TG=122 mg/dL), has high blood pressure (135/79), overweight (BMI=29.2), with no history of smoking, diabetes, atrial fibrillation, chronic kidney disease, rheumatoid arthritis or family CVD.

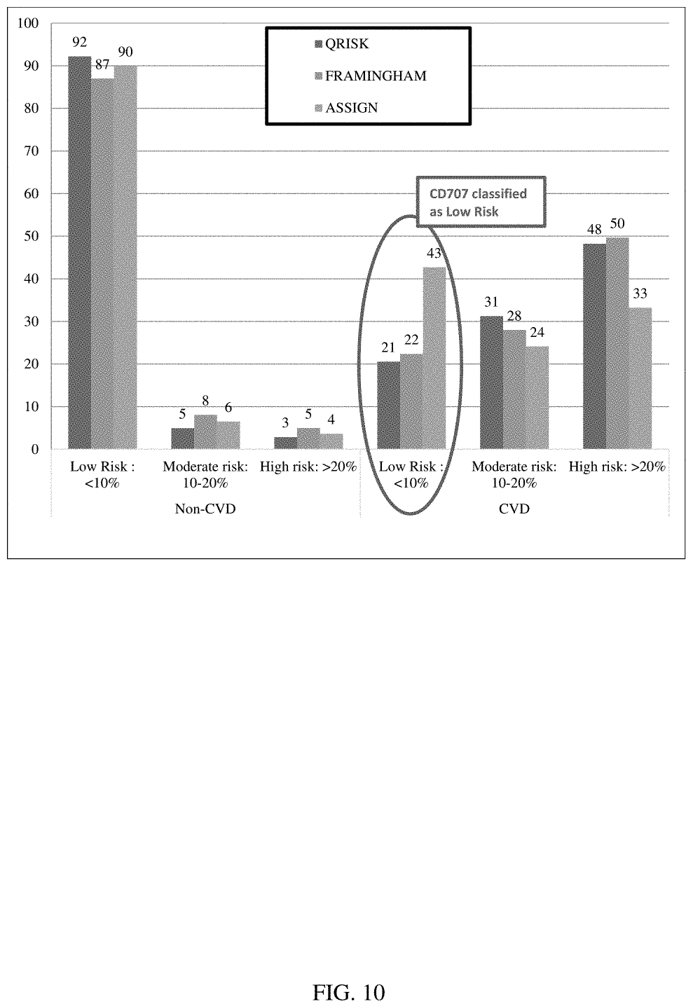

The three established models identified participant X as Low Risk (FIG. 10) with a less than 10% chance of having a CVD event in the next 10 years (ASSIGN RS=4.23, Framingham RS=4.42, and QRISK2 RS=2.79). Whilst, both the Discriminant Analysis (DA RS=3.98) and Logistic Regression (LG RS=1.76) identified participant X as clearly CVD.

Participant X had a myocardial infarction (MI) and was diagnosed with multivessel coronary artery disease (CAD) requiring three Percutaneous Coronary Interventions (PCI, formerly known as angioplasty with stents). Each PCI can cost up to TT$120,000.00 (US$18,750.00) per procedure. The cost for treating CVD increases each year in Trinidad and Tobago and was last estimated at 6 billion at the end of 2016. The ability to determine a person's risk of having a CVD is important towards prevention and treatment which consequently contributes towards lowering the healthcare fiscal burden.

For participant X, clearly, the established models (Framingham, ASSIGN, and QRISK2) cannot be used for classification since they are underestimating CVD risk compared to the proprietary models (Discriminant Analysis and Logistic Regression).

Risk Model Discrimination by Ethnicity

Five models were used to predict the risk of having a CVD event. For the established models (ASSIGN, Framingham, and QRISK2), the risk was estimated as the risk of having a CVD event within the next 10 years. For the models developed from the TT2015 sample (Logistic and Discriminant), the risk was estimated as being currently at risk for CVD. The best-established model and calculated models were determined for each Ethnicity. Distribution of all participants in TT2015 sample was separated based on Ethnicity and then by four sub-categories viz. Clearly Non-CVD (Non-CVD), Non-CVD persons in the Non-Differentiating range (Non-differentiating —Non-CVD), CVD persons in the Non-Differentiating range (Non-differentiating —CVD) and clearly CVD (CVD). Preference was given to models that had the lowest percentage of participants in the Non-Differentiating CVD range and the highest percentage of persons in the CVD category because it is more important to not misclassify CVD persons.

For the Afro-Trinbagonians the Logistic Regression and ASSIGN Model performed the best at discriminating CVD participants in the Non-Discriminating range (see Table 13). The Logistic only had 9% of CVD participants in the Non-Differentiating range and was able to correctly identify 88% of all CVD participants. While the established ASSIGN model was able to correctly identify 50% of CVD participants as CVD and had the lowest percentage of CVD participants in the Non-Differentiating range (20%).

For Indo-Trinbagonians the Logistic Regression and QRISK2 models also had better CVD discrimination. The Logistic Regression only had 8% of CVD participants in the Non-Differentiating range and was able to correctly identify 90% of all CVD participants. While the established QRISK2 model was able to correctly identify 72% of CVD participants as CVD and had the lowest percentage of CVD participants in the Non-Differentiating range (13%).

The Discriminant model performed the best for Mixed-Trinbagonians CVD participants (Table 13) compared to Afro- and Indo-Trinbagonians. The Discriminant had 11% of CVD participants in the Non-Differentiating range and was able to correctly identify 89% of all CVD participants. While the established ASSIGN model was the best at discriminating for Mixed-Trinbagonians, it performed almost marginally better than the QRISK2 model which had a higher percentage of CVD persons in the Non-Differentiating range (31% vs. 34%).

TABLE 13
Discrimination power of each of the five models tested in this study by Ethnicity

| Ethnicity | Category | ARS | FRS | QRS | LG | DS |
|---|---|---|---|---|---|---|
| Afro-Trinbagonian | Non-CVD | 81 | 88 | 91 | 76 | 79 |
| | Non-CVD in Non-discriminating range | 13 | 7 | 7 | 22 | 20 |
| | CVD in Non-discriminating range | 20 | 33 | 30 | 8 | 27 |
| | CVD | 50 | 50 | 53 | 79 | 73 |
| Indo-Trinbagonian | Non-CVD | 80 | 88 | 82 | 76 | 79 |
| | Non-CVD in Non-discriminating range | 13 | 10 | 10 | 23 | 21 |
| | CVD in Non-discriminating range | 24 | 31 | 13 | 9 | 16 |
| | CVD | 50 | 46 | 72 | 88 | 84 |
| Mixed-Trinbagonian | Non-CVD | 80 | 88 | 86 | 76 | 72 |
| | Non-CVD in Non-discriminating range | 11 | 10 | 10 | 19 | 25 |
| | CVD in Non-discriminating range | 31 | 37 | 34 | 10 | 11 |
| | CVD | 51 | 38 | 54 | 88 | 89 |

ARS: ASSIGN Risk Score Model
FRS: Framingham Risk Score Model
QRS: QRISKII Risk Score Model
LG: Logistic Regression Model
DS: Discriminant Model

Operational embodiments disclosed herein may be embodied directly in hardware, in a software module executed by a processor, or in a combination of the two. A software module may reside in RAM memory, flash memory, ROM memory, EPROM memory, EEPROM memory, registers, hard disk, a removable disk, a CD-ROM, a DVD disk, or any other form of storage medium known in the art. An exemplary storage medium is coupled to the processor such the processor may read information from, and write information to, the storage medium. In the alternative, the storage medium may be integral to the processor. The processor and the storage medium may reside in an ASIC or may reside as discrete components in another device.

Furthermore, the one or more versions may be implemented as a method, apparatus, or article of manufacture using standard programming and/or engineering techniques to produce software, firmware, hardware, or any combination thereof to control a computer to implement the disclosed embodiments. Non-transitory computer readable media may include but are not limited to magnetic storage devices (e.g., hard disk, floppy disk, magnetic strips), optical disks (e.g., compact disk (CD), digital versatile disk (DVD)), smart cards, and flash memory devices (e.g., card, stick). Those skilled in the art will recognize many modifications may be made to this configuration without departing from the scope of the disclosed embodiments.

The previous description of the disclosed embodiments is provided to enable any person skilled in the art to make or use the present disclosure. Various modifications to these embodiments will be readily apparent to those skilled in the art, and the generic principles defined herein may be applied to other embodiments without departing from the spirit or scope of the disclosure. Thus, the present disclosure is not intended to be limited to the embodiments shown herein but is to be accorded the widest scope consistent with the principles and novel features disclosed herein.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is in no way intended that an order be inferred, in any respect. This holds for any possible non-express basis for interpretation, including: matters of logic with respect to arrangement of steps or operational flow; plain meaning derived from grammatical organization or punctuation; the number or type of embodiments described in the specification.

It will be apparent to those of ordinary skill in the art that various modifications and variations may be made without departing from the scope or spirit. Other embodiments will be apparent to those skilled in the art from consideration of the specification and practice disclosed herein. It is intended that the specification and examples be considered as exemplary only, with a true scope and spirit being indicated by the following claims.

What is claimed is:

1. A method for deriving a personalized cardiovascular disease (CVD) risk assessment for an individual, via a computing system, wherein the computing system comprises:
(a) a processor operable to control the computing system,
(b) data storage operatively coupled to the processor, wherein the data storage is configured to store a plurality of background data, a plurality of phenotypic measurement data, and combinations thereof,
(c) an input/output device operatively coupled to the processor, wherein the input/output device is configured to receive a plurality of data for transmission to the processor, wherein the input/output device is configured to transmit a plurality of data generated by the processor,
(d) a profile classification component operatively coupled to the processor and controlled in part by the processor, wherein the profile classification component is configured to determine a profile classification for the individual,
(e) a risk factor profile component operatively coupled to the processor and controlled in part by the processor, wherein the risk factor profile component is configured to generate a risk factor profile for the individual, and
(f) a risk prediction component operatively coupled to the processor and controlled in part by the processor, wherein the risk prediction component is configured to generate a personalized CVD risk assessment for the individual, the method comprising:
receiving, via the input/output device, a plurality of selected background data associated with the individual and transmitting at least a portion of the plurality of background data to the profile classification component;
receiving, via the input/output device, a plurality of phenotypic measurement data associated with the individual and transmitting at least a portion of the phenotypic measurement data to the profile classification component;
determining, by the profile classification component, based on at least a portion of the background information and the phenotypic measurement data, a profile classification for the individual and generating profile classification data therefrom;
transmitting, via the input/output device, the profile classification data to the risk factor profile component;
determining, by the risk factor profile component, based on the profile classification data, at least one individual risk factor and associated risk factor weight to be included in the personalized CVD risk assessment, and generating a personalized risk factor profile for the individual;
selectively integrating, by the risk prediction component, at least a portion of the background data and phenotypic measurement data in accordance with the personalized risk factor profile to generate integrated risk data; and
subjecting, by the risk prediction component, the integrated risk data to risk prediction function to generate a personalized CVD risk assessment for the individual.

2. The method of claim 1, wherein the selected background data is selected from the group consisting of sociodemographic data, personal and family medical history data, medication usage, diet and physical activity data, behavioral data, and combinations thereof.

3. The method of claim 1, wherein the phenotypic measurement data is selected from the group consisting of biomedical record data, health care record data, bioassay data, medical imaging data, blood analysis data, metabolic test data, physiologic data, and combinations thereof.

4. The method of claim 1, further comprising receiving, via the input/output device, a plurality of genetic data associated with the individual and transmitting at least a portion of the genetic data to the profile classification component;
wherein the data storage is operable to store a plurality of genetic data; and
wherein the profile classification for the individual is determined based on at least a portion of the background information, the phenotypic measurement data, and the genetic data.

5. The method of claim 4, wherein the genetic data is selected from the group consisting of genotype data, structural variant data, sequence data, and combinations thereof.

6. The method of claim 4, wherein at least a portion of the background data, phenotypic data, and genetic data are selectively integrated in accordance with the personalized risk factor profile to generate integrated risk data.

7. The method of claim 1, wherein the computing system further comprises a cardiac health recommendation component operatively connected to the processor and controlled in part by the processor, wherein the cardiac health recommendation component is configured to generate a plurality of cardiac health recommendations, the method further comprising:
transmitting, via the input/output device, the personalized CVD risk assessment to the cardiac health recommendation component;
determining, by the cardiac health recommendation component, based on the personalized CVD risk assessment, at least one cardiac health regime.

8. The method of claim 1, wherein the integrated risk data is subjected to a logistic regression function to generate a personalized CVD risk assessment for the individual.

9. The method of claim 1, wherein the integrated risk data is subjected to a discriminant analysis function to generate a personalized CVD risk assessment for the individual.

10. A system for deriving a personalized cardiovascular disease (CVD) risk assessment for an individual, the system comprising
a processor operable to control the system;
data storage operatively coupled to the processor, wherein the data storage is configured to store a plurality of background data, a plurality of phenotypic measurement data, and combinations thereof;
an input/output device operatively coupled to the processor, wherein the input/output device is configured to receive a plurality of data for transmission to the processor, wherein the input/output device is configured to transmit a plurality of data generated by the processor;
a profile classification component operatively coupled to the processor and controlled in part by the processor, wherein the profile classification component is configured to determine a profile classification for the individual;
a risk factor profile component operatively coupled to the processor and controlled in part by the processor, wherein the risk factor profile component is configured to generate a risk factor profile for the individual; and
a risk prediction component operatively coupled to the processor and controlled in part by the processor, wherein the risk prediction component is configured to generate a personalized CVD risk assessment for the individual;
wherein the input/output device is operable to:
receive a plurality of selected background data associated with the individual and transmit at least a portion of the plurality of background data to the profile classification component; and
receive a plurality of phenotypic measurement data associated with the individual and transmit at least a portion of the phenotypic measurement data to the profile classification component;
wherein the profile classification component is operable to:
receive at least a portion of the selected background data and the phenotypic measurement data from the input/output device; and
determine, based on at least a portion of the background information and the phenotypic measurement data, a profile classification for the individual and generating profile classification data therefrom; and
transmit, via the input/output device, the profile classification data to the risk factor profile component;
wherein the risk factor profile component is operable to:
receive the profile classification data from the input/output device; and
determine, based on the profile classification data, at least one individual risk factor and associated risk factor weight to be included in the personalized CVD risk assessment, and generate a personalized risk factor profile for the individual; and
transmit, via the input/output device, the personalized risk factor profile to the risk prediction component;
wherein the risk prediction component is operable to:
receive the personalized risk factor profile from the input/output device; and
selectively integrate at least a portion of the background data and phenotypic measurement data in accordance with the personalized risk factor profile to generate integrated risk data;
subject the integrated risk data to risk prediction function to generate a personalized CVD risk assessment for the individual.

11. The system of claim 10, wherein the selected background data is selected from the group consisting of sociodemographic data, personal and family medical history data, medication usage, diet and physical activity data, behavioral data, and combinations thereof.

12. The system of claim 10, wherein the phenotypic measurement data is selected from the group consisting of biomedical record data, health care record data, bioassay data, medical imaging data, blood analysis data, metabolic test data, physiologic data, and combinations thereof.

13. The system of claim 10, wherein the input/output device is further operable to receive a plurality of genetic data associated with the individual and transmit at least a portion of the genetic data to the profile classification component;
wherein the data storage is operable to store a plurality of genetic data; and
wherein the profile classification component is operable to determine a profile classification for the individual based on at least a portion of the background information, the phenotypic measurement data, and the genetic data.

14. The system of claim 13, wherein the genetic data is selected from the group consisting of genotype data, structural variant data, sequence data, and combinations thereof.

15. The system of claim 13, wherein the risk prediction component is operable to selectively integrate at least a portion of the background data, phenotypic data, and genetic data in accordance with the personalized risk factor profile to generate integrated risk data.

16. The system of claim 10, further comprising a cardiac health recommendation component operatively connected to the processor and controlled in part by the processor, wherein the cardiac health recommendation component is configured to generate a plurality of cardiac health recommendations;
wherein the input/output device is operable to transmit the personalized CVD risk assessment to the cardiac health recommendation component; and
wherein the cardiac health recommendation component is operable to determine, based on the personalized CVD risk assessment, at least one cardiac health regime.

17. The system of claim 10, wherein the risk prediction component is operable to subject the integrated risk data to a logistic regression function to generate a personalized CVD risk assessment for the individual.

18. The system of claim 10, wherein the risk prediction component is operable to subject the integrated risk data to a discriminant analysis function to generate a personalized CVD risk assessment for the individual.
